(19) United States
(12) Patent Application Publication
(10) Pub. No.: US 2002/0009444 A1
(43) Pub. Date: Jan. 24, 2002

(54) Intrathecal administration of rituximab for treatment of central nervous system lymphomas

(75) Inventor: Antonio J. Grillo-Lopez, Rancho Santa Fe, CA (US)

Correspondence Address: Pillsbury Winthrop LLP, Intellectual Property Group, East Tower, Ninth Floor, 1100 New York Avenue, N.W., Washington, DC, 20005-3918, US

(73) Assignee: IDEC Pharmaceuticals Corporation, San Diego, CA (US)

(21) Appl. No.: 09/840,872

(22) Filed: Apr. 25, 2001

Related U.S. Application Data
(60) Provisional application No. 60/199,365, filed on Apr. 25, 2000.

(51) Int. Cl. A61K 51/10; A61K 39/395; A61K 51/04; A61K 31/525; A61K 31/475

(52) U.S. Cl. 424/142.1; 424/1.65; 514/251; 424/1.49; 514/283

(57) ABSTRACT

This invention describes methods of using anti-B cell antibodies, preferably anti-CD20 antibodies, and most preferably Rituximab, to treat B cell lymphomas of the brain, especially primary central nervous system lymphomas (PCNSLs), and to prevent meningeal relapse. The antibodies can be administered intrathecally alone, or in combination with other chemotherapeutics, such as methotrexate, or other anti-B cell antibodies to treat PCNSL in both immunocompromised and non-immunocompromised patients. These antibodies can also be used to diagnose patients with CNS lymphoma, especially in immunocompromised patients.

### CROSS REFERENCE TO RELATED APPLICATION

This application claims priority from U.S. Provisional Ser. No. 60/199,365, filed Apr. 25, 2000, and is incorporated herein in its entirety by reference.

### FIELD OF THE INVENTION

This invention describes methods of using antibodies to a B cell target, e.g., anti-CD20, anti-CD21, anti-CD22, anti-CD23, anti-CD40 or anti-CD37 antibodies, and preferably an anti-CD20 antibody, and still more preferably Rituximab, to treat and/or prevent central nervous system lymphomas and to prevent meningeal relapse. These anti-B cell antibodies can be used alone or in combination with other antibodies, e.g., antibodies to T cells involved in B cell activation such as anti- CD40L, or other therapies (e.g., chemotherapy or radiotherapy).

### BACKGROUND OF THE INVENTION

I. Anti-CD20 Antibodies

CD20 is a cell surface antigen expressed on more than 90% of B-cell lymphomas and does not shed or modulate in the neoplastic cells (McLaughlin et al., J. Clin. Oncol. 16: 2825-2833 (1998b)). Anti-CD20 antibodies have been prepared for use both in research and therapeutics. One anti-CD20 antibody is the monoclonal B1 antibody (U.S. Pat. No. 5,843,398). Anti-CD20 antibodies have also been prepared in the form of radionuclides for treating B-cell lymphoma (e.g., ¹³¹I-labeled anti-CD20 antibody), as well as a ⁸⁹Sr-labeled form for the palliation of bone pain caused by prostate and breast cancer metastasises (Endo, Gan To Kagaku Rvoho 26: 744-748 (1999)).

A murine monoclonal antibody, 1F5, (an anti-CD20 antibody) was reportedly administered by continuous intravenous infusion to B cell lyniphoma patients. However, extremely high levels (>2 grams) of 1F5 were reportedly required to deplete circulating tumor cells, and the results were described as “transient” (Press et al., Blood 69: 584-591 (1987)). A potential problem with using monoclonal antibodies in therapeutics is those non-human monoclonal antibodies (e.g., murine monoclonal antibodies) typically lack human effector functionality, e.g., they are unable to, inter alia, mediate complement dependent lysis or lyse human target cells through antibody-dependent cellular toxicity or Fc-receptor mediated phagocytosis. Furthermore, non-human monoclonal antibodies can be recognized by the human host as a foreign protein; therefore, repeated injections of such foreign antibodies can lead to the induction of immune responses leading to harmful hypersensitivity reactions. For murine-based monoclonal antibodies, this is often referred to as a Human Anti-Mouse Antibody response, or “HAMA” response. Additionally, these “foreign” antibodies can be attacked by the immune system of the host such that they are, in effect, neutralized before they reach their target site.

A. Rituximab

Rituximab (also known as Rituxan®, MabThera® and IDEC-C2B8) was the first FDA-approved monoclonal antibody and was developed at IDEC Pharmaceuticals (see U.S. Pat. Nos. 5,843,439; 5,776,456 and 5,736,137). Rituximab is a chimeric, anti-CD20 monoclonal (MAb) recommended for treatment of patients with low-grade or follicular B-cell non-Hodgkin's lymphoma (McLaughlin et al., Oncology (Huntingt) 12: 1763-1777 (1998a); Leget et al., Curr. Opin. Oncol. 10: 548-551 (1998)). In Europe, Rituximab has been approved for therapy of relapsed stage lII/IV follicular lymphoma (White et aL, Pharm. Sci. Technol. Today 2: 95-101 (1999)). Other disorders treated with Rituximab include follicular centre cell lymphoma (FCC), mantle cell lymphoma (MCL), diffuse large cell lymphoma (DLCL), and small lymphocytic lymphoma/chronic lymphocytic leukemia (SLL/CLL) (Nguyen et al., 1999)). Rituximab has exhibited minimal toxicity and significant therapeutic activity in low-grade non-Hodgkin's lymphomas (NHL) in phase I and II clinical studies (Berinstein et al., Ann. Oncol. 9: 995-1001 (1998)).

Rituximab, which was used alone to treat B cell NHL at weekly doses of typically 375 mg/M² for four weeks with relapsed or refractory low-grade or follicular NHL, was well tolerated and had significant clinical activity (Piro et al., Ann. Oncol. 10: 655-61 (1999); Nguyen et al., Eur. J. Haematol. 62: 76-82 (1999); and Coiffier et al., Blood 92: 1927-1932 (1998)). However, up to 500 mg/M² of four weekly doses have also been administered during trials using the antibody (Maloney et al., Blood 90: 2188-2195 (1997)). Rituximab also has been combined with chemotherapeutics, such as CHOP (e.g., cyclophosphamide, doxorubicin, vincristine and prednisone), to treat patients with low-grade or follicular B-cell non-Hodgkin's lymphoma (Czuczman et al, J. Clin. Oncol. 17: 268-76 (1999); and McLaughlin et al., Oncology (Huntingt) 12: 1763-1777 (1998)).

II. CD40 and CD40L

CD40 is expressed on the cell surface of mature B cells, as well as on leukemic and lymphocytic B cells, and on Hodgkin's and Reed-Sternberg (RS) cells of Hodgkin's Disease (HD) (Valle et al, Eur. J. himunol. 19: 1463-1467 (1989); and Gruss et al., Leuk. Lymphoma 24: 393-422 (1997)). CD40 is a B cell receptor leading to activation and survival of normal and malignant B cells, such as non-Hodgkin's follicular lymphoma (Johnson et al., Blood 82: 1848-1857 (1993)). Signaling through the CD40 receptor protects immature B cells and B cell lymphomas from IgM-or fas-induced apoptosis (Wang et al., J. hnmunol. 155: 3722-5 (1995)). Similarly, mantel cell lymphoma cells have a high level of CD40, and the addition of exogenous CD40L enhanced their survival and rescued them from fludarabine-induced apoptosis (Clodi et al., Brit. J. Haematol. 103: 217-9 (1998)). In contrast, others have reported that CD40 stimulation may inhibit neoplastic B cell growth both in vitro (Funakoshi et al., Blood 83: 2787-2794 (1994)) and in vivo (Murphy et al., Blood 86: 1946-1953 (1995)).

Anti-CD40 antibodies administered to mice purportedly increased the survival of mice with human B-cell lymphomas (Funakoshi et al., (1994); and Tutt et al, J. Immunol. 161: 3176-3185 (1998)). Methods of treating neoplasms, including B cell lymphomas and EBV-induced lymphomas using anti-CD40 antibodies to inhibit CD40-CD40L interaction, is described in U.S. Pat. No. 5,674,492 (1997) and International PCT Application WO 95/17202, herein incorporated by reference in their entirety. CD40 signals reportedly have also been associated with a synergistic interaction with CD20 (Ledbetter et al, Circ. Shock 44: 67-72 (1994)). Additional references describing preparation and use of anti-CD40 antibodies include U.S. Pat. Nos. 5,874,085 (1999), 5,874,082 (1999), 5,801,227 (1998), and 5,674,492 (1997) incorporated herein by reference in their entirety.

A CD40 ligand, gp39 (also called CD40 ligand or CD40L), is expressed on activated, but not resting, CD4⁺Th cells (Spriggs et al., J. Exp. Med. 176: 1543-1550 (1992); Lane et al., Eur. J. Immunol. 22: 2573-2578 (1992); and Roy et al., J. Inimunol. 151: 1-14 (1993)). Both CD40 and CD40L have been cloned and characterized (Stamenkovi et al., EMBO J. 8: 1403-1410 (1989); Armitage et al., Nature 357: 80-82(1992); Lederman etal., J.Exp. Med. 175: 1091-1101 (1992); and Hollenbaugh et aL, EMBO J. 11: 4313-4321 (1992)). Cells transfected with the CD40L gene and expressing the CD40L protein on their surface can trigger B cell proliferation, and together with other stimulatory signals, can induce antibody production (Armitage et al., (1992)). CD40L may play an important role in the cell contact-dependent interaction of tumor B-cells (CD40⁺) within the neoplastic follicles or Reed-Sternberg cells (CD40⁺) in Hodgkin's Disease areas (Carbone et al., Am. J. Pathol. 147: 912-22 (1995)).

Anti-CD40L monoclonal antibodies have been effectively used to inhibit the induction of murine AIDS (MAIDS) in LP-BM5-infected mice (Green et al., Virology 241: 260-268 (1998)). Anti-CD40 antibodies have also been prepared to prevent or treat antibody-mediated diseases, such as allergies and autoimmune disorders as described in U.S. Pat. No. 5,874,082 (1999). Anti-CD40 antibodies reportedly have been combined with anti-CD20 antibodies yielding an additive effect in inhibiting growth of non-Hodgkin's B cell lymphomas in cell culture (Benoit et al., (1996) Immunopharmnacology 35: 129-139 (1996)). In vivo studies in mice demonstrated that anti-CD20 antibodies were more efficacious than anti-CD40 administered individually in promoting the survival of mice bearing some, but not all, lymphoma lines (Funakoshi et al., J. Immunother. Emphasis Tumor Immunol. 19: 93-101 (1996)). Anti-CD19 is also effective in vivo in the treatment of two syngeneic mouse B cell lymphomas, BCL1 and A31 (Tutt et al. (1998)).

Antibodies to CD40L have been described for use to treat disorders associated with B cell activation (European Patent No. 555,880 (1993)). Anti-CD40L antibodies include monoclonal antibodies 3E4, 2H5, 2H8, 4D9-8, 4D9-9, 24-31, 24-43, 89-76 and 89-79, as described in U.S. Pat. No. 5,747,037 (1998), and anti-CD40L antibodies described in U.S. Pat. No. 5,876,718 (1999) used to treat graft-versus-host-disease.

III. Central Nervous System Cancers and Their Treatment

A. Primary Central Nervous System Lymyhomas (PCNSLs)

Primary central nervous system lymphoma (PCNSL) is defined as a lymphoma limited to the brain and brain stem without systemic disease. It is a term applied to non-Hodgkin's lymphoma (NHL) arising in and confined to the central nervous system (CNS). In the past, this tumor has also been referred to as a microglioma, a reticulum cell sarcoma or a perivascular sarcoma. Today, however, its lymphatic origin is now well established.

PCNSL was formerly a rare tumor accounting for only 0.5 to 1.2% of all intracranial neoplasms, usually associated with congenital, acquired or iatrogenic immunodeficiency states, such as Wiskott-Aldrich syndrome or immunosuppression arising from renal transplantation. The highest incidence of PCNSL is reported in patients with acquired immunodeficiency syndrome (AIDS), in whom it is seen in 1.9 to 6% (DeAngelis et al., “Primary Central Nervous System Lymphoma,” IN CANCER: PRINCIPLES & PRACTICE OF ONCOLOGY 2233-2242 (DeVita et al., eds. 1997). However the incidence of PCNSL is increasing in patients who are not immunocompromised.

Both systemic and primary CNS non-Hodgkin's lymphomas occur in people with AIDS (Kramer et al., Cancer 80: 2469-2477 (1997)). Moreover, a substantial difference exists between AIDS and non-AIDS patients with PCNSL clinically, diagnostically and prognostically (Fine et al., Ann. Intern. Med. 119: 1093-1104 (1993)).

HIV-related PCNSL is an aggressive non-Hodgkin's lymphoma (NHL) and is exclusively contained within the CNS. Most HIV-related PCNSLs are histologically classified as either diffuse, large cell or large cell immunoblastic lymphomas of B cell origin. Additionally, the origin of PCNSL remains controversial, with questions persisting as to whether it arises from intracranial transformation of infiltrating non-malignant lymphocytes or whether peripheral neoplastic cells migrate to and bind exclusively within the CNS (Moses et al., 1999).

The optimal treatment for PCNSL also has not been defined (Reni et al., Ann. Oncol. 8: 227-234 (1997); and Lesser et al., Cancer Treat. Rev. 19: 261-281 (1993)). PCNSL arising as a complication from AIDS, due to its location and multifocality, is generally not surgically resectable. Typical therapy has been cranial radiation involving external beam radiotherapy at a dose of 4,000-5,000 cGy. Although clinical and radiographic improvement is rapid, median survival is only two to five months. Whole brain irradiation and adjuvant chemotherapy consisting of preirradiation CHOP (e.g., cyclophosphamide, doxorubicin, vincristine and prednisone) and post-irradiation cytarabine has also been used, however many of the patients nevertheless die (O'Neill et al., Int'l J. Radiation Oncol. Biol. Phys. 33: 663-673 (1995)). Combined cytarabine (e.g., ARA-C), methotrexate and cranial radiotherapy has been reported as more effective than radiotherapy alone (Abrey et al., J. Clin. Oncol. 16: 859-63 (1998)). A combination of high dosage methotrexate, leucovorin, thiotepa, vincristine and dexamethasone also has been reported as effective for treating non-immunocompromised patients (Sandor et al., J. Clin. Oncol. 16: 3000-3006 (1998)). Combined methotrexate and cytarabine administration using an Ommaya reservoir has been reported effective for treating combined intraocular lymphoma with CNS involvement (Valluri et al., Retina 15: 125-9 (1995)); new treatment modalities for such intraocular lymphomas are useful, as ocular involvement occurs in 20% of patients with PCNSL (Monjour et al., Rev. Neurol. (Paris) 148: 589-600 (1992)). Unfortunately, severe cognitive deficits are reported with these intensive therapies due to iatrogenic leukoencephalopathy. Retrospective data suggests decreased risk of dementia occurs when chemotherapy is employed prior to radiation therapy (Fine et al., Annals Intern. Med. 119: 1093-1104 (1993); and Blay et al., J. Clin. Oncol. 16: 864-871 (1998)). Other studies have proposed the use of chemotherapy alone to treat PCNSL. The effects of chemotherapy purportedly can be enhanced using agents that increase permeability of the chemotherapeutic agents across the blood-brain barrier (Cheng et al., Cancer 82: 1946-51 (1998).

Nevertheless, despite these treatment options, median survival remains fixed at approximately 40 months (Abrey et al., J. Clin. Onc. 16: 859-863 (1998)). Moreover, these therapies are associated with definite, fixed risks in delayed neurotoxicity which is severe in 100% of patients older than 60 years of age (Abrey et al, “Combination chemotherapy in primary central nervous system lymphoma,” (abstract) Proc. Am. Soc. Clin. Onc. (1999)). Also, involvement of the CNS complicates 5-29% of systemic NHL cases and is associated with an extremely grave prognosis (Fine et al., Ann. Intern. Med. 119: 1093-1104 (1993)); and van Besien et al., Blood 91: 1178-1184 (1998)).

B. Other CNS Cancers and Their Treatments

Other CNS cancers include metastasises of NHL to the brain, such as leptomeningeal metastasises (LM). LM has been treated with intra-Ommaya injection of methotrexate and ¹¹¹Indium-diethylenetriamine pentaacetic acid (¹¹¹In-DTPA) with mixed results (Mason et al., Neurology 50: 438-444 (1998)). Cytarabine and thiotepa have also been combined with irradiation to treat LM (Schabet et al., Nervenarzt 63: 317-27 (1992)). LM has also been diagnosed in a patient with Stage IV Hodgkin's disease (HD); the patients reportedly were successfully treated with whole brain irradiation and intrathecal methotrexate (Orlowski et al., Cancer 53: 1833-1835 (1984)).

Current therapies for primary brain tumors, brain metastasises, and leptomeningeal carcinomatosus, including the use of monoclonal antibodies, have been inadequate or have little therapeutic activity. Linking monoclonal antibodies to protein toxins has been proposed as an agent for treating CNS cancers (Youle, Semin. Cancer Biol. 7: 65-70 (1996)). For example, immunotoxins, such as anti-CD7 ricin A chain (DA7), have been reported as in animal models of LM (Herrlinger et al, J. Neurooncol. 40: 1-9 (1998)). LMB-7 (a single chain immunotoxin constructed from a murine monoclonal antibody B3 and a truncated Pseudomonas exotoxin PE38) purportedly has been used to treat neoplastic meningitis in a mouse model (Pastan et al, Proc. Nat'l Acad. Sci. USA 92: 2765-2769 (1995)).

IV. Drug Delivery to the Brain

Delivery of therapeutics to the brain to treat brain tumors of any type has posed a hurdle because of the blood-brain barrier (BBB). Methods of treating brain cancer include: (1) surgical management when possible; (2) whole brain radiotherapy; (3) corticosteroids in non-immunocompromised patients; and (4) chemotherapy which has the ability to penetrate the BBB. Administration of chemotherapeutics can be any infusion route, such as brain interstitial infusion (Shin et aL, J. Neurosurz. 82: 1021-1029 (1995)) or intrathecal administration. Osmotic BBB disruption procedures have also been designed to treat intracerebral tumors (Kroll et al., Neurosurgery 42: 1083-99 (1998)).

Other agents that penetrate the BBB have also been developed. For example, lipophilic delivery vectors (e.g., procarbazine), as well as high dosage CNS penetrable agents (e.g., high dose methotrexate) are recommended for treating PCNSL (DeAngelis et al., 1997). Recently, the use of the monoclonal antibody OX26, which allows for vector-mediated drug delivery through the BBB in rats, has been proposed for use in targeting brain cancers (Partridge et al., Pharm. Res. 15: 576-82 (1998)). The OX26 MAb can reportedly be utilized in delivering conjugated peptide radiopharmaceuticals to the brain (Deguchi et al., Bioconjug. Chem. 10: 32-37 (1999)). Other monoclonal antibodies purportedly have been prepared as brain drug-delivery vectors, which are directed against cell surface receptors (e.g., the transferrin receptor or the insulin receptor) on the brain capillary endothelium, which comprises the BBB in vivo (Wu et al., Drug. Metabl. Dispos. 26: 937-9 (1998)). Immunoliposomes (antibody-directed liposomes) have also been prepared which purportedly can deliver the anti-neoplastic agent, daunomycin, to a rat brain (Huwyler et aL, Proc. Nat'l Acad. Sci. USA 93: 14164-14169 (1996)). Biomolecular lipophilic complexes have also been described, which purportedly can deliver active agents to mammalian brains (U.S. Pat. No. 5,716,614).

Therefore, not withstanding what has been previously reported in the literature, there exists a need for improved diagnosis and treatment for PCNSL and other B cell lymphomas of the brain. Moreover, to the best of the inventor's knowledge, no one has proposed administering an anti-CD20 antibody intrathecally alone, or in combination with other anti-cancer agents or antibodies (e.g., anti-CD40 or anti-CD40L antibodies), to treat central nervous system lymphomas and meningeal relapse.

### OBJECTS AND SUMMARY OF THE INVENTION

It is an object of the instant invention to provide a method to treat or prevent meningeal relapse in a subject with lymphoma comprising the step of administering a therapeutically effective amount of an antibody to a B cell target, e.g., anti-CD22, anti-CD21, anti-CD23, anti-CD37, anti-CD40, anti-CD20 antibody or fragment thereof. Another object of the invention is to provide a method of treating a central nervous system (CNS) lymphoma which comprises the step of administering a therapeutically effective amount of an antibody directed to a B cell or an antibody that affects B cell activation, e.g., anti-CD21, anti-CD22, anti-CD23, anti-CD40, anti-CD40L, or anti-CD20 antibody or fragment thereof. The CNS lymphomas targeted for treatment include: primary CNS lymphoma, (PCNSL) leptomeningeal metastasises (LM), or Hodgkin's Disease with CNS involvement.

It is a particular object of the invention to use anti-B cell antibodies which are human antibodies, humanized antibodies, bispecific antibodies or chimeric antibodies for treatment of CNS lymphoma. For example, anti-CD20, anti-CD21, anti-CD22, anti-CD23, anti-CD40 or anti-CD40L antibody fragments, such as Fab, Fab′ and F(ab′)₂, are also contemplated for use in treating CNS lymphomas.

A more preferred object of the invention is to use Rituximab as an anti-CD20 antibodies used for treating CNS lymphomas. The anti-CD20 antibody can be administered, preferably intraventricularly or intrathecally at a dosage of about 10 mg to about 375 mg/M² per week for four weeks.

Another object of the invention is to administer an anti-CD20 antibody in combination with any one or more of the following (1) an anti-CD40 antibody, or another B cell binding antibody, (2) a CD40L antagonist, (3) a chemotherapeutic agent or agents, and/or (4) an anti-B cell antibody for treatment of CNS lymphomas.

It is a further object of the invention to link the anti-B cell antibody, e.g., anti-CD20 antibody or an antibody to other B cell targets identified infra, to a radioisotope for purposes of therapy or diagnosis of CNS lymphoma. The anti-CD20 antibody or another anti-B cell antibody can be linked to ²¹¹At, ²¹²Bi, ⁶⁷Cu, ¹²³I, ¹³¹I, ¹¹¹In, ³²p, ²¹²Pb, ¹⁸⁶Re, ¹⁸⁸Re, ¹⁵³Sm, ^{99m}Tc, or ⁹⁰Y, and if administered for a therapeutic purpose, it is administered to a subject in a radioimmunotherapeutically effective amount.

Another object of the invention is a method of diagnosing a CNS lymphoma, such as PCNSL, in a subject comprising the steps of: (A) administering an antibody to a B cell anti-CD20 antibody or anti-CD20 antibody fragment bound to a detectable label to a subject; and (B) detecting the localization of said label.

The composition administered for treating a CNS lymphoma can be combined with or linked to a brain blood barrier (BBB) permeability enhancing reagent.

### DETAILED DESCRIPTION OF THE INVENTION

I. Definitions

By “CNS lymphoma” is meant any B cell lymphoma of the central nervous system (CNS). This can include Hodgkin's Disease (ND) lymphomas, non-Hodgkin's lymphoma (NHL), leptomeningeal metastasises and primary CNS lymphoma (“PCNSL”).

As used herein, the term “antibody” is meant to refer to complete, intact antibodies, and Fab fragments, Fv, scFv and F(ab)₂ fragments thereof. Complete, intact antibodies include monoclonal antibodies, such as murine monoclonal antibodies (mAb), chimeric antibodies, primatized antibodies, humanized antibodies and human antibodies. The production of antibodies and the protein structures of complete, intact antibodies, Fab fragments and F(ab)₂ fragments and the organization of the genetic sequences that encode such molecules are well known and are described, for example, in Harlow et al., ANTIBODIES: A LABORATORY MANUAL, Cold Spring Harbor Laboratory, Cold Spring Harbor, N.Y. (1988) which is incorporated herein by reference. The antibodies (e.g., anti-CD20, anti-B cell antibodies etc.) can be in the form as complete, intact antibodies or fragments in the form of immunotoxins or bispecific antibodies.

By “anti-CD40 antibody” is intended to include immunoglobulins and fragments thereof, which are specifically reactive with a CD40 protein or peptide thereof or a CD40 fusion protein. Anti-CD40 antibodies can include human antibodies, chimeric antibodies, bispecific antibodies and humanized antibodies.

By “B cell surface marker” or “B cell target” or “B cell antigen” is meant an antigen expressed on the surface of a B cell which can be targeted with an antagonist that binds therein. Exemplary B cell surface markers include CD10, CD14, CD20, CD21, CD22, CD23, CD24, CD37, CD53, CD72, CD73, CD74, CD75, CD76, CD77, CD78, CD79a, CD79b, CD80, CD81, CD82, CD83, CDw84, CD85 and CD86 leukocyte surface markers. A B cell surface marker of particular interest is one preferentially expressed on B cells relative to other non-B cell tissues of a mammal and may be expressed on both precursor B cells and mature B cells. In a preferred embodiment, the B cell marker will use CD19, CD20 or CD22, which are found on B cells throughout differentiation of the lineage from the stem cell stage up to a point just prior to terminal differentiation into plasma cells. The most preferred B cell marker is CD20.

An “antibody to a B cell” or “B cell antibody” is an antibody that specifically binds an antigen on a B cell, e.g. those identified supra.

A “B cell antagonist” is a molecule which, upon binding to a B cell surface marker, destroys or depletes B cells in a mammal and/or interferes with one or more B cell functions, e.g. by reducing or preventing a humoral response elicited by the B cell. The antagonist preferably is able to deplete B cells (i.e. reduce circulating B cell levels) in a mammal treated therewith. Such depletion may be achieved via various mechanisms such antibody-dependent cell mediated cytotoxicity (ADCC) and/or complement dependent cytotoxicity (CDC), inhibition of B cell proliferation and/or induction of B cell death (e.g. via apoptosis). Antagonists included within the scope of the present invention include antibodies, synthetic or native sequence peptides and small molecule antagonists which bind to the B cell marker, optionally conjugated with or fused to a cytotoxic agent. The preferred antagonist comprises an antibody, more preferably a B cell depleting antibody.

By “anti-CD40L antibody” is intended to include immunoglobulins and fragments thereof, which are specifically reactive with a CD40L protein or peptide thereof or a CD40L fusion protein. Anti-CD40L antibodies can include human antibodies, chimeric antibodies, bispecific antibodies and humanized antibodies.

By “anti-CD20 antibody” is intended to include immunoglobulins and fragments thereof, which are specifically reactive with CD20 or a peptide thereof. Anti-CD20 antibodies can include human antibodies, humanized antibodies, chimeric antibodies and bi- or tri-specific antibodies. A preferred anti-CD20 antibody is Rituximab.

By “B cell depleting antibody” is meant any antibody (including chimeric and humanized antibodies) or fragment thereof or immunotoxin containing which, when administered therapeutically, depletes the number of B cells from the subject to which the antibody was administered. Such B cell depleting antibodies can include, for example, but are not limited to antibodies that bind any of the B cell antigens identified above, and include preferably anti-CD20 antibodies, anti-CD19 antibodies, anti-CD22 antibodies, anti-CD38 antibodies (e.g., OKT10 antibody, see, Flavell et al., Int. J. Cancer 62: 337-44 (1995)), and anti-major histocompatibility complex (MHC) II antibodies (see Illidge et al., Blood 94: 233-43 (1999)). B cell depleting antibodies preferably will be anti-CD20 antibodies. B cell depleting antibodies can in a radioactive form linked to a therapeutic isotope, as an immunotoxin linked to a toxic agent, the whole antibody or fragments thereof (e.g., Fab′), as well as chimeric antibodies and humanized antibodies of B cell depleting antibodies.

By “anti-CD19 antibody” is meant any antibody or fragment thereof or immunotoxin which recognizes and binds to a CD19 antigen expressed on a B cell. Preferred anti-CD19 antibodies are those that can therapeutically deplete a subject of B cells or effect a B cell in a manner making it more sensitive to other agents or reducing the cell's life span. Specific anti-CD19 antibodies include, but are not limited to, monoclonal antibody HD37 (see Ghetie et al, Clin. Cancer Res. 5: 3920-7 (1999)), monoclonal antibody B43 or its derived single chain Fv (VFS191) (Li et al., Cancer Immunol. Immunother. 47: 121-30 (1998)), monoclonal murine antibody HD37 (Stone et al., Blood 88: 1188-97 (1996)), and single chain Fv (scFv) antibody fragment FVS192 (Bejcek et aL, Cancer Res. 55: 2346-51 (1995)).

By “anti-CD22 antibody” is meant any antibody or fragment thereof or immunotoxin which recognizes and binds to a CD22 antigen expressed on a B cell. Preferred anti-CD22 antibodies are those that can therapeutically deplete a subject of B cells or effect a B cell in a manner making it more sensitive to other agents or reducing the cell's life span. Specific anti-CD22 antibodies include, but are not limited to, humanized anti-CD22 antibody hLL2 (Behr et al., Clin. Cancer Res. 5: 3304s-14s (1999)), monoclonal antibody OM124 (Bolognesi et al., Br. J. Haematol. 101: 179-88 (1998)), and anti-CD22 IgG₁ antibody RFB4 and immunotoxins thereof (Mansfield et al., Bioconjug. Chem. 7: 557-63 (1996)).

By “bispecific antibody” is meant an antibody molecule with one antigen-binding site specific for one antigen, and the other antigen-binding site specific for another antigen.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcyRIII only, whereas monocytes express FcyRI, FcyRII and FcyRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in US Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector functions. Preferably, the cells express at least FcyRiH and carry out ADCC effector function. Examples of human leukocytes which mediate ADCC include peripheral blood mononuclear cells (PBMC), natural killer () cells, monocytes, cytotoxic T cells and neutrophils; with PBMCs and NK cells being preferred.

The terms “Fc receptor” or “FCR” are used to describe a receptor that binds to the Fc region of an antibody.

The preferred FcR is a native sequence human FcR. Moreover, a preferred FcR is one which binds an IgG antibody (a gamma receptor) and includes receptors of the FcyRI, FcyRII, and Fcy RIII subclasses, including allelic variants and alternatively spliced forms of these receptors. FcyRII receptors include FcyRIIA (an “activating receptor”) and FcyRIIB (an “inhibiting receptor”), which have similar amino acid sequences that differ primarily in the cytoplasmic domains thereof. Activating receptor FcyRIIA contains an immunoreceptor tyrosine-based activation motif (ITAM) in its cytoplasmic domain. Inhibiting receptor FcyRIIB contains an immunoreceptor tyrosine-based inhibition motif (ITIM) in its cytoplasmic domain. (see Daeron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FCR” herein. The term also includes the neonatal receptor, FcRn, which is responsible for the transfer of maternal IgGs to the fetus (Guyer et al., J. nmnunol. 117:587 (1976) and Kim et al., J. Immunol. 24:249 (1994)).

“Complement dependent cytotoxicity” or “CDC” refer to the ability of a molecule to lyse a target in the presence of complement. The complement activation pathway is initiated by the binding of the first component of the complement system (Clq) to a molecule (e.g. an antibody) complexed with a cognate antigen. To assess complement activation, a CDC assay, e.g. as described in Gazzano-Santoro et al., J. Immunol. Methods 202:163 (1996), may be performed.

“Growth inhibitory” antagonists are those which prevent or reduce proliferation of a cell expressing an antigen to which the antagonist binds. For example, the antagonist may prevent or reduce proliferation of B cells in vitro and/or in vivo.

Antagonists which “induce apoptosis” are those which induce programmed cell death, e.g. of a B cell, as determined by standard apoptosis assays, such as binding of annexin V, fragmentation of DNA, cell shrinkage, dilation of endoplasmic reticulum, cell fragmentation, and/or formation of membrane vesicles (called apoptotic bodies).

“Antibody fragments” comprise a portion of an intact antibody, preferably comprising the antigen-binding or variable region thereof. Examples of antibody fragments include Fab, Fab′, F(ab′)2, and Fv fragments; diabodies; linear antibodies; single-chain antibody molecules; and multi specific antibodies formed from antibody fragments.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a P-sheet configuration, connected by three hypervariable regions, which form loops connecting, and in some cases forming part of, the (3 sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Papain digestion of antibodies produces two identical antigen-binding fragments, called “Fob” fragments, each with a single antigen-binding site, and a residual “Fc” fragment, whose name reflects its ability to crystallize readily. Pepsin treatment yields an F(ab′2 fragment that has two antigen-binding sites and is still capable of crosslinking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CHI) of the heavy chain. Fab′ fragments differ from Fab fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear at least one free thiol group. F(ab′)Z antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (x) and lambda (k), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG 1, IgG2, IgG3, IgG4, IgA, and IgA2. The heavy-chain constant domains that correspond to the different classes of antibodies are called a, 8, s, y, and R, respectively. The subunit structures and three-dimensional configurations of different classes of immunoglobulins are well known.

“Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. For a review of scFv see Pluckthun in The Pharmacology of Monoclonal Antibodies, vol. 113, Rosenburg and Moore, eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen binding sites. Diabodies are described more fully in, for example, EP 404,097; WO 93/11161; and Hollinger et al., Proc. Nad. Acad. Sci. USA, 90:6444.-6448 (1993).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et aL, J MoL BioL, 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. NatL. Acad. Sci. USA, 81:6851-6855 (1984)). Chimeric antibodies of interest herein include “primatized” antibodies comprising variable domain antigen binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the amino acid residues of an antibody which are responsible for antigen-binding. The hypervariable region comprises amino acid residues from a “complementarity determining region” or “CDR” (e.g. residues 24-34 (LI), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined. An antagonist “which binds” an antigen of interest, e.g. a B cell surface marker, is one capable of binding that antigen with sufficient affinity and/or avidity such that the antagonist is useful as a therapeutic agent for targeting a cell expressing the antigen.

Examples of antibodies which bind the CD20 antigen include: “C2B8” which is now called “rituximab” (“RITUXAN®”) (U.S. Pat. No. 5,736,137, expressly incorporated herein by reference); the yttrium-[90]-labeled 2138 murine antibody designated “Y2B8” (U.S. Pat. No. 5,736,137, expressly incorporated herein by reference); murine IgG2a “131 ” optionally labeled with 1311 to generate the “131 I-B1” antibody (BEXXARTM) (U.S. Pat. No. 5,595,721, expressly incorporated herein by reference); murine monoclonal antibody “1F5” (Press et al Blood 69(2):584-591 (1987)); “chimeric 2H7” antibody (U.S. Pat. No. 5,677,180, expressly incorporated herein by reference); and monoclonal antibodies L27, G28-2, 93-1133, B-C1 or NU-B2 available from the International Leukocyte Typing Workshop (Valentine et al., In: Leukocyte Typing III (McMichael, Ed., p. 440, Oxford University Press (1987)). Examples of antibodies which bind the CD19 antigen include the anti-CD19 antibodies in Hekman et al., Cancer Immunol. Immunother. 32:364-372 (1991) and Vlasveld et al. Cancer Immunol. Immunother. 40:37-47(1995); and the B4 antibody in Kiesel et al. Leukemia Research 11, 12: 1119 (1987).

The terms “rituximab” or “RITUXAN®” herein refer to the genetically engineered chimeric murine/human monoclonal antibody directed against the CD20 antigen and designated “C2B8” in U.S. Pat. No. 5,736,137, expressly incorporated herein by reference. The antibody is an IgG, kappa immunoglobulin containing murine light and heavy chain variable region sequences and human constant region sequences. Rituximab has a binding affinity for the CD20 antigen of approximately 8.OnM.

An “isolated” antagonist is one which has been identified and separated and/or recovered from a component of its natural environment. Contaminant components of its natural environment are materials which would interfere with diagnostic or therapeutic uses for the antagonist, and may include enzymes, hormones, and other proteinaceous or nonproteinaceous solutes. In preferred embodiments, the antagonist will be purified (1) to greater than 95% by weight of antagonist as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antagonist includes the antagonist in situ within recombinant cells since at least one component of the antagonist's natural environment will not be present. Ordinarily, however, isolated antagonist will be prepared by at least one purification step. “Mammal” for purposes of treatment refers to any animal classified as a mammal, including humans, domestic and farm animals, and zoo, sports, or pet animals, such as dogs, horses, cats, cows, etc. Preferably, the mammal is human.

“Treatment” refers to both therapeutic treatment and prophylactic or preventative measures. Those in need of treatment include those already with the disease or disorder as well as those in which the disease or disorder is to be prevented. Hence, the mammal may have been diagnosed as having the disease or disorder or may be predisposed or susceptible to the disease.

The expression “therapeutically effective amount” refers to an amount of the antagonist which is effective for preventing, ameliorating or treating the autoimmune disease in question. The term “immunosuppressive agent” as used herein for adjunct therapy refers to substances that act to suppress or mask the immune system of the mammal being treated herein. This would include substances that suppress cytokine production, downregulate or suppress self-antigen expression, or mask the MHC antigens.

Examples of such agents include 2-amino-6-aryl-5-substituted pyrimidines (see U.S. Pat. No. 4,665,077, the disclosure of which is incorporated herein by reference); azathioprine; cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as glucocorticosteroids, e.g., prednisone, methylprednisolone, and dexamethasone; cytokine or cytokine receptor antagonists including anti-interferon-y, -(3, or-a antibodies, anti-tumomecrosis factor-a antibodies, anti-tumornecrosis factor-(i antibodies, anti-interleukin-2 antibodies and anti-IL-2 receptor antibodies; anti-LFA-1 antibodies, including anti-CD 1la and anti-CD18 antibodies; anti-L3T4 antibodies; heterologous anti-lymphocyte globulin; pan-T antibodies, preferably antiCD3 or anti-CD4/CD4a antibodies; soluble peptide containing a LFA-3 binding domain (WO 90/08187 published Jul. 26, 1990); streptokinase; TGF-0; streptodornase; RNA or DNA from the host; FK506; RS-61443; deoxyspergualin; rapamycin; T-cell receptor (Cohen et aL, U.S. Pat. No. 5,114,721); T-cell receptor fragments (Offner et al., Science 251: 430-432 (1991); WO 90/11294; laneway, Nature, 341: 482 (1989); and WO 91/01133); and T cell receptor antibodies (EP 340,109) such as TLOB9.

The term “cytotoxic agent” as used herein refers to a substance that inhibits or prevents the function of cells and/or causes destruction of cells. The term is intended to include radioactive isotopes (e.g. I¹³¹, Y⁹⁰, Ar²¹¹, P³², Re¹⁸⁸, Re¹⁸⁶, Sm¹⁵³, B²¹² and others), chemotherapeutic agents, and toxins such as small molecule toxins or enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as thiotepa and cyclosphosphamide (CYTOXANTM); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembiehin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromoinycins, dactinomycin, daunorubicin, detorubicin, 6-diazo-5-oxo-L-norleucine, doxorubicin, epirubicin, esorubicin, idambicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofrran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOLO, Bristol-Myers Squibb Oncology, Princeton, N.J.) and doxetaxel (TAXOTEW, Rh6ne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4 hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-a and-0; mullerian-inhibiting substance; mouse gonadotropin-associated peptide; inhibin; activin; vascular endothelial growth factor; integrin; thrombopoietin (TPO); nerve growth factors such as NGF-P; platelet growth factor; transforming growth factors (TGFs) such as TGF-a and TGF-0; insulin-like growth factor-I and -II; erythropoietin (EPO); osteoinductive factors; interferons such as interferon-a, -P, and -y; colony stimulating factors (CSFs) such as macrophage-CSF (M-CSF); granulocyte-macrophage-CSF (GM-CSF); and granulocyte-CSF (GCSF); interleukins (ILs) such as IL-1, IL-1a, IL-2, IL-3, IL-4, IL-5, IL-6, IL-7, IL-8, IL-9, IL-11, IL-12, IL-15; a tumor necrosis factor such as TNF-a or TNF-P; and other polypeptide factors including LIF and kit ligand (KL). As used herein, the term cytokine includes proteins from natural sources or from recombinant cell culture and biologically active equivalents of the native sequence cytokines.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wihnan, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, (3-lactam-containing prodrugs, optionally substituted phenoxyacetamide-containing prodrugs or optionally substituted phenylacetamide-containing prodrugs, 5 fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

A “liposome” is a small vesicle composed of various types of lipids, phospholipids and/or surfactant which is useful for delivery of a drug (such as the antagonists disclosed herein and, optionally, a chemotherapeutic agent) to a mammal. The components of the liposome are commonly arranged in a bilayer formation, similar to the lipid arrangement of biological membranes. The term “package insert” is used to refer to instructions customarily included in commercial packages of therapeutic products, that contain information about the indications, usage, dosage, administration, contraindications and/or warnings concerning the use of such therapeutic products.

By “therapeutically effective amount” or “prophylactically effective amount” or “dose effective amount” is meant an amount of an agent which inhibits the progression of a CNS lymphoma. Such inhibition can be a fall response resulting in undetectable presence of the lymphoma or a partial response. It is especially advantageous to formulate parenteral compositions in dosage unit form for ease of administration and uniformity of the dosage. “Dosage unit form,” as used herein, refers to physically discrete units suited as unitary dosages for the mammalian subjects to be treated; each unit containing a predetermined quantity of active compound is calculated to produce the desired therapeutic effect in association with the required pharmaceutical carrier. The specification for the dosage unit forms of the invention are dictated by and directly dependent on: (A) the unique characteristics of the active compound and the particular therapeutic effect to be achieved; and (B) the limitations inherent in the art of compounding such an active compound for the treatment of sensitivity in individuals.

By “radioimmunotherapeutically effective amount” is meant that amount of an anti-CD20 antibody linked to a radioactive isotope which when administered to a subject for the treatment of a CNS lymphoma, causes the CNS lymphoma to fully or partially regress. Typically, any of the antibodies discussed are administered in a dosage range of 300-1500 mg/m³.

By “pharmaceutical excipient” refers to any inert substance that is combined with an active drug, agent, or antigen for preparing an agreeable or convenient dosage form.

By “immunogenicity” is meant the ability of a targeting protein or therapeutic moiety to elicit an immune response (e.g., humoral or cellular) when administered to a subject.

II. Production of Antagonists

The methods and articles of manufacture of the present invention use, or incorporate, an antagonist which binds to a B cell surface marker, e.g., CD20, CD19, CD21, CD22, CD40 et al. Accordingly, methods for generating such antagonists will be described here. The B cell surface marker or cytokine to be used for production of, or screening for, antagonist(s) may be, e.g., a soluble form of the antigen or a portion thereof, containing the desired epitope. Alternatively, or additionally, cells expressing the B cell surface marker at their cell surface can be used to generate, or screen for, antagonist(s). Other forms of the B cell surface marker useful for generating antagonists will be apparent to those skilled in the art. Preferably, the B cell surface marker is the CD19 or CD20 antigen.

While the preferred antagonist is an antibody, antagonists other than antibodies are contemplated herein. For example, the antagonist may comprise a small molecule antagonist optionally fused to, or conjugated with, a cytotoxic agent (such as those described herein). Libraries of small molecules may be screened against the B cell surface marker of interest herein in order to identify a small molecule which binds to that antigen. The small molecule may further be screened for its antagonistic properties and/or conjugated with a cytotoxic agent.

The antagonist may also be a peptide generated by rational design or by phage display (see, e.g., W098/35036 published Aug. 13, 1998). In one embodiment, the molecule of choice may be a “CDR mimic” or antibody analogue designed based on the CDRs of an antibody. While such peptides may be antagonistic by themselves, the peptide may optionally be fused to a cytotoxic agent so as to add or enhance antagonistic properties of the peptide.

A description follows as to exemplary techniques for the production of the antibody antagonists used in accordance with the present invention.

Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOC12, or R1N═C═NR, where R and RI are different alkyl groups. Animals are immunized against the antigen, immunogenic conjugates, or derivatives by combining, e.g., 100 pg or 5 wg of the protein or conjugate (for rabbits or mice, respectively) with 3 volumes of Freund's complete adjuvant and injecting the solution intradermally at multiple sites. One month later the animals are boosted with ⅕to 1/10the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal Antibodies

Monoclonal antibodies are obtained from a population of substantially homogeneous antibodies, Le., the individual antibodies comprising the population are identical except for possible naturally occurring mutations that may be present in minor amounts. Thus, the modifier “monoclonal” indicates the character of the antibody as not being a mixture of discrete antibodies. For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell [Goding, Monoclonal Antibodies: Principles and Practice, pp. 59-103 (Academic Press, 1986)].

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse human heteromyeloma cell lines also have been described for the production of human monoclonal antibodies [Kozbor, J. Immunol., 133:3001 (1984); Brodeur et al., Monoclonal Antibody Production Techniques and Applications, pp. 51-63 (Marcel Dekker, Inc., New York, 1987)].

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subdloned by limiting dilution procedures and grown by standard methods (Goding, Monoclonal Antibodies: Principles and Practice, pp.59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subdlones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Phickthun, Immunol. Revs., 130:151-188 (1992).

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J Mol. Biol., 222:581-597 (1991) describe the isolation of murine and human antibodies, respectively, using phage libraries. Subsequent publications describe the production of high affinity (DM range) human antibodies by chain shuffling (Marks et al., BiolTechnology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al, Proc. Natl Acad. Sci. USA, 81:6851 (1984)), or by covalently joining to the immunoglobulin coding sequence all or part of the coding sequence for a non-immunoglobulin polyp eptide. Typically such non-immuno globulin polyp eptides are substituted for the constant domains of an antibody, or they are substituted for the variable domains of one antigen-combining site of an antibody to create a chimeric bivalent antibody comprising one antigen-combining site having specificity for an antigen and another antigen combining site having specificity for a different antigen.

Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al, Nature, 332:323-327 (1988); Verhoeyen et aL, Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al, J. Immunol, 151:2296 (1993); Chothia et al., J Mol. Biol, 196:901 (1987)). Another method uses a particular framework region derived from the consensus sequence of all human antibodies of a particular subgroup of light or heavy chains. The same framework may be used for several different humanized antibodies (Carter et aL, Proc. Nad. Acad. Sci. USA, 89:4285 (1992); Presta et aL, J. Immunol, 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three-dimensional immunoglobulin models are commonly available and are familiar to those skilled in the art. Computer programs are available which illustrate and display probable three-dimensional conformational structures of selected candidate timmunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al, Proc. Mad. Acad. Sci. USA, 90:2551 (1993); Jakobovits et al., Nature, 362:255-258 (1993); Bruggermann et aL, Year in Immuno., 7:33 (1993); and U.S. Pat. Nos. 5,591,669, 5,589,369 and 5,545,807. Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g. Johnson, Kevin S. and Chiswell, David J., Current Opinion in Structural Biology 3:564-571(1993). Several sources of V-gene segments can be used for phage display. Clackson et al., Nature, 352: 624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J Mol. Biol. 222:581-597 (1991), or Griffith et al., EMBO J. 12:725-734 (1993). See, also, U.S. Pat. Nos. 5,565,332 and 5,573,905. Human antibodies may also be generated by in vitro activated B cells (see U.S. Pat. Nos. 5,567,610 and 5,229,275).

Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-Sli fragments can be directly recovered from E. coli and chemically coupled to form F(ab′)2 fragments [Carter et al., Bio/Technology 10:163-167 (1992)]. According to another approach, F(ab′)2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. 5,641,870 for example. Such linear antibody fragments may be monospecific or bispecific.

Bispecific Antibodies

Bispecific antibodies are antibodies that have binding specificities for at least two different epitopes. Exemplary bispecific antibodies may bind to two different epitopes of the B cell surface marker. Other such antibodies may bind a first B cell marker and further bind a second B cell surface marker. Alternatively, an anti-B cell marker binding arm may be combined with an arm which binds to a triggering molecule on a leukocyte such as a T-cell receptor molecule (e.g. CD2 or CD3), or Fc receptors for IgG (FcyR), such as FcyRI (CD64), FcyRII (CD32) and FcyRIII (CD16) so as to focus cellular defense mechanisms to the B cell. Bispecific antibodies may also be used to localize cytotoxic agents to the B cell. These antibodies possess a B cell marker-binding arm and an arm which binds the cytotoxic agent (e.g. saporin, anti-interferon-a, vinca alkaloid, ricin A chain, methotrexate or radioactive isotope hapten). Bispecific antibodies can be prepared as full length antibodies or antibody fragments (e.g. F(ab′)Z bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J, 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CHI) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid imnunoglobulin heavy chain with a first binding specificity in one arm, and a hybrid immunoglobulin heavy chain light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et aL, Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229:81 (1985) describe a procedure wherein intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)2 molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain.

Accordingly, the VH arid VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. Immunol., 152:5368 (1994). Antibodies with more than two valencies are contemplated. For example, trispecific antibodies can be prepared. Tutt et aL J. Immunol. 147: 60 (1991).

III. Conjugates and Other Modifications of the Antagonist

The antagonists used in the methods or included in the articles of manufacture herein are optionally conjugated to a cytotoxic agent. Chemotherapeutic agents useful in the generation of such antagonist-cytotoxic agent conjugates have been described above.

Conjugates of an antagonist and one or more small molecule toxins, such as a calicheamicin, a maytansine (U.S. Pat. No. 5,208,020), a trichothene, and CC1065 are also contemplated herein. In one embodiment of the invention, the antagonist is conjugated to one or more maytansine molecules (e.g. about 1 to about 10 maytansinemolecules per antagonist molecule). Maytansine may, for example, be converted to May-SS-Me which may be reduced to May-SH3 and reacted with modified antagonist (Chari et al. Cancer Research 52: 127-131 (1992)) to generate a maytansinoid-antagonist conjugate.

Alternatively, the antagonist is conjugated to one or more calicheamicin molecules. The calicheamicin family of antibiotics are capable of producing double-stranded DNA breaks at sub-picomolar concentrations. Structural analogues of calicheamicin which may be used include, but are not limited to, ′yJ1, a21, a31, N-acetyl-yl′, PSAG and 011 (Hinman et al. Cancer Research 53: 3336-3342 (1993) and Lode et al. Cancer Research 58: 2925-2928 (1998)).

Enzymatically active toxins and fragments thereofwhich can be used include diphtheria A chain, nonbinding active fragments of diphtheria toxin, exotoxin A chain (from Pseudomonas aeruginosa), ricin A chain, abrin A chain, modeccin A chain, alpha-sarcin, 41euritesfordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates antagonist conjugated with a compound with nucleolytic activity (e.g. a ribonuclease or a DNA endonuclease such as a deoxyribonuclease; DNase). A variety of radioactive isotopes are available for the production of radioconjugated antagonists. Examples include At²¹¹,I¹²⁵, Re¹⁸⁸, In¹¹¹, Tc^{99m}, pb²¹², Y⁹⁰, Re¹⁸⁶, Sm¹⁵³, Cu⁶⁷, I¹³¹, P⁵², Bi²¹² and radioactive isotopes of Lu. Conjugates of the antagonist and cytotoxic agent may be made using a variety of bifunctional protein coupling agents such as N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aidehydes (such as glutareldehyde), bis azido compounds (such as bis (p-azidobenzoyl) hexanediamine), bis-diazonium derivatives (such as bis-(pdiazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antagonist. See W094/11026. The linker may be a “cleavable linker” facilitating release of the cytotoxic drug in the cell. For example, an acid-labile linker, peptidase-sensitive linker, dimethyl linker or disulfide-containing linker (Chari et aL Cancer Research 52: 127-131 (1992)) may be used. Alternatively, a fusion protein comprising the antagonist and cytotoxic agent may be made, e.g. by recombinant techniques or peptide synthesis.

In yet another embodiment, the antagonist may be conjugated to a “receptor” (such streptavidin) for utilization in tumor pretargeting wherein the antagonist-receptor conjugate is administered to the patient, followed by removal of unbound conjugate from the circulation using a clearing agent and then administration of a “ligand” (e.g. avidin) which is conjugated to a cytotoxic agent (e.g. a radionucleotide). The antagonists of the present invention may also be conjugated with a pro drug-activating enzyme which converts a pro drug (e.g. a peptidyl chemotherapeutic agent, see W081/01145) to an active anti-cancer drug. See, for example, WO 88/07378 and U.S. Pat. No. 4,975,278.

The enzyme component of such conjugates includes any enzyme capable of acting on a prodrug in such a way so as to covert it into its more active, cytotoxic form. Enzymes that are useful in the method of this invention include, but are not limited to, alkaline phosphatase useful for converting phosphate-containing prodrugs into free drugs; arylsulfatase useful for converting sulfate containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate cleaving enzymes such as li-galactosidase and neuraminidase useful for converting glycosylated prodrugs into free drugs; (3-lactamase useful for converting drugs derivatized with (3-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328: 457-458 (1987)). Antagonist-abzyme conjugates can be prepared as described herein for delivery of the abzyme to a tumor cell population.

The enzymes of this invention can be covalently bound to the antagonist by techniques well known in the art such as the use of the heterobifunctional crosslinking reagents discussed above. Alternatively, fusion proteins comprising at least the antigen binding region of an antagonist of the invention linked to at least a functionally active portion of an enzyme of the invention can be constructed using recombinant DNA techniques well known in the art [see, e.g., Neuberger et al., Nature, 312: 604-608 (1984)].

Other modifications of the antagonist are contemplated herein. For example, the antagonist may be linked to one of a variety of nonproteinaceous polymers, e.g., polyethylene glycol, polypropylene glycol, polyoxyalkylenes, or copolymers of polyethylene glycol and polypropylene glycol. The antagonists disclosed herein may also be formulated as liposomes. Liposomes containing the antagonist are prepared by methods known in the art, such as described in Epstein et al., Proc. Mad. Acad Sci. USA, 82:3688 (1985); Hwang et al., Proc. Natl Acad. Sci. USA, 77:4030 (1980); U.S. Pat. Nos. 4,485,045 and 4,544,545; and W097/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody of the present invention can be conjugated to the liposomes as described in Martin et al., J Biol. Chem. 257: 286-288 (1982) via a disulfide interchange reaction. A chemotherapeutic agent is optionally contained within the liposome. See Gabizon et al. J National Cancer Inst.81(19)1484 (1989). Amino acid sequence modification(s) of protein or peptide antagonists described herein are contemplated. For example, it may be desirable to improve the binding affinity and/or other biological properties of the antagonist.

Amino acid sequence variants of the antagonist are prepared by introducing appropriate nucleotide changes into the antagonist nucleic acid, or by peptide synthesis. Such modifications include, for example, deletions from, and/or insertions into and/or substitutions of, residues within the amino acid sequences of the antagonist. Any combination of deletion, insertion, and substitution is made to arrive at the final construct, provided that the final construct possesses the desired characteristics. The amino acid changes also may alter post-translational processes of the antagonist, such as changing the number or position of glycosylation sites.

A useful method for identification of certain residues or regions of the antagonist that are preferred locations for mutagenesis is called “alanine scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antagonist variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antagonist with an N-terminal methionyl residue or the antagonist fused to a cytotoxic polypeptide. Other insertional variants of the antagonist molecule include the fusion to the N-or C-terminus of the antagonist of an enzyme, or a polypeptide which increases the serum half-life of the antagonist.

Another type of variant is an amino acid substitution variant. These variants have at least one amino acid residue in the antagonist molecule replaced by different residue. The sites of greatest interest for substitutional mutagenesis of antibody antagonists include the hypervariable regions, but FR alterations are also contemplated.

Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened.

TABLE 1

| Original | Exemplary | Preferred |
|---|---|---|
| Residue | Substitutions | Substitutions |
| Ala (A) | val; leu; ile | val |
| Arg (R) | lys; gin; asn | lys |
| Asn (N) | gin; his; asp, lys; arg | gln |
| Asp (D) | glu; asn | glu |
| Cys (C) | ser; ala | ser |
| Gin (Q) | asn; glu | asn |
| Glu (E) | asp; gin | asp |
| Gly (G) | ala | ala |
| His (H) | asn; gin; lys; arg | arg |
| Ile (I) | leu; val; met; ala; | ICU |
| | phe; norleucine | |
| Lea (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| TIP (W) | tyr; phe | tyr |
| Tyr (Y) | trp; phe; thr; ser | phe |
| Val (V) | ile; leu; met; phe; | ICU |
| | ala; norleucine | |

Substantial modifications in the biological properties of the antagonist are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gIn, his, lys, arg;

(5) residues that influence chain orientation: gly, pro; and

(6) aromatic: trp, tyr, phe.

Non-conservative substitutions will entail exchanging a member of one of these classes for another class.

Any cysteine residue not involved in maintaining the proper conformation of the antagonist also may be substituted, generally with serine, to improve the oxidative stability of the molecule and prevent aberrant crosslinking. Conversely, cysteine bond(s) may be added to the antagonist to improve its stability (particularly where the antagonist is an antibody fragment such as an Fv fragment).

A particularly preferred type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or in additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antagonist alters the original glycosylation pattern of the antagonist. By altering is meant deleting one or more carbohydrate moieties found in the antagonist, and/or adding one or more glycosylation sites that are not present in the antagonist.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Addition of glycosylation sites to the antagonist is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antagonist (for O-linked glycosylation sites).

Nucleic acid molecules encoding amino acid sequence variants of the antagonist are prepared by a variety of methods known in the art. These methods include, but are not limited to, isolation from a natural source (in the case of naturally occurring amino acid sequence variants) or preparation by oligonucleotide-mediated (or site directed) mutagenesis, PCR mutagenesis, and cassette mutagenesis of an earlier prepared variant or a non-variant version of the antagonist.

It may be desirable to modify the antagonist of the invention with respect to effector function, e.g. so as to enhance antigen-dependent cell-mediated cyotoxicity (ADCC) and/or complement dependent cytotoxicity (CDC) of the antagonist. This may be achieved by introducing one or more amino acid substitutions in an Fc region of an antibody antagonist. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J Exp Med. 176:1191-1195 (1992) and Shopes, B. J Immunol. 148:2918-2922 (1992). Homodimeric antibodies with enhanced anti-tumor activity may also be prepared using heterobifunetional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989).

To increase the serum half life of the antagonist, one may incorporate a salvage receptor binding epitope into the antagonist (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

IV. Pharmaceutical Formulations

Therapeutic formulations of the antagonists used in accordance with the present invention are prepared for storage by mixing an antagonist or antagonists having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt forming counter-ions such as sodium; metal complexes (e.g. Zn-protein complexes); and/or non-ionic surfactants such as TWEEN™, PLURONICS™ or polyethylene glycol (PEG).

Exemplary anti-CD20 antibody formulations are described in W098/56418, expressly incorporated herein by reference. This publication describes a liquid multidose formulation comprising 40 mg/mL rituximab, 25 mM acetate, 150 mM trehalose, 0.9% benzyl alcohol, 0.02% polysorbate 20 at pH 5.0 that has a minimum shelf life of two years storage at 2-8° C. Another anti-CD20 formulation of interest comprises I Omg/mL rituximab in 9.0 mg/mL sodium chloride, 7.35 mg/mL sodium citrate dihydrate, 0.7 mg/mL polysorbate 80, and Sterile Water for Injection, pH 6.5. Lyophilized formulations adapted for subcutaneous administration are described in W097/04801. Such lyophilized formulations may be reconstituted with a suitable diluent to a high protein concentration and the reconstituted formulation may be administered subcutaneously to the mammal to be treated herein.

The formulation herein may also contain more than one active compound zi.; necessary for the particular indication being treated, preferably those with complementary activities that do not adversely affect each other. For example, it may be desirable to further provide a cytotoxic agent, chemotherapeutic agent, cytokine or immunosuppressive agent (e.g. one which acts on T cells, such as cyclosporin or an antibody that binds T cells, e.g. one which binds LFA-1). The effective amount of such other agents depends on the amount of antagonist present in the formulation, the type of disease or disorder or treatment, and other factors discussed above. These are generally used in the same dosages and with administration routes as used hereinbefore or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations maybe prepared. Suitable examples of sustained-release preparations include semipermeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOTTM (injectable microspheres composed of lactic acid glycolic acid copolymer and leuprolide acetate), and poly-D-(-)-3-hydroxybutyric acid.

The formulations to be used for in vivo administration must be sterile. This is readily accomplished by filtration through sterile filtration membranes.

V. Methods and Compositions for Administering Anti-B Cell Antibodies

A. Methods for Administering Anti-B Cell Antibodies

Methods for administering anti-B cell antibodies for use in treating CNS lymphomas can be intravenous (iv), oral or intraperitoneal. However, the preferred method of administering anti-B cell antibodies, e.g., anti-CD20 antibodies, or immunogenically active fragments thereof for treating central nervous system lymphomas or related conditions is by intrathecal administration. Intrathecal administration will preferably be by Ommaya reservoir, but can also be administered via a lumbar puncture or intraventrically. The anti-B cell antibodies can be administered by either the same route in combination with another drug; the secondary agent alternatively can be administered by a separate route. Additionally, the anti-B cell antibodies contemplated may be administered prior to or post cranial irradiation.

Alternatively, the blood brain barrier (BBB) can be disrupted, followed by administration of drugs intra-arterially. Anti-B cell antibodies such as anti-CD20 antibodies that bind B cells, or anti-CD40L antibodies which inhibit B cells, can be administered intra-arterially either alone or in combination with other agents (e.g., anti-CD40 antibodies, other anti-B cell antibodies, methotrexate, cyclophosphamide, procarbazine and dexamethasone). Methods of disrupting the BBB include those described in Kroll et al., Neurosurgery 42: 1083-99 (1998) and Dahlborg et al., Cancer J. Sci. Am. 2: 166 (1996).

As noted, the anti-B cell antibodies, e.g., anti-CD20 antibodies, such as Rituximab, or therapeutically effective fragments thereof (e.g., Fab, Fab′ or F(ab′)₂) will be administered alone or in combination with one or more additional active agents. Additional active agents can include other chemotherapeutics such as leucovorin, CHOP, methotrexate, cytarabine, thiotepa or vincristine such as those described previously. Anti-B cell antibodies or therapeutically effective fragments thereof can also be administered in combination with agents which inhibit the interaction between CD40 and its ligand, CD40L. CD40/CD40L inhibitors can include anti-CD40 antibodies or fragments thereof, anti-CD40L antibodies or fragments thereof and peptide mimetics of either CD40 or CD40L. Anti-CD20 antibodies in particular can also be administered with other anti-B cell antibodies, such as anti-CD19, anti-CD22, anti-CD38 and anti-MHCII antibodies. Moreover, anti-CD20 antibodies can be administered alone, in combination with other antibodies or in combination with other treatment modalities (e.g., chemotherapy and radiation therapy), as well as combinations thereof.

These active agents (e.g., anti-CD20 antibodies, such as Rituximab) can be in a pharmaceutically effective carrier or vector. Vectors can include lipophilic vectors (e.g., procarbazine) or immunolipophilic vectors such as those described by Huwyler et al., Proc. Nat′l Acad. Sci. USA 93: 14164-14169 (1996) and U.S. Pat. No. 5,716,614). Alternatively, the active agent can be linked to vectors which target receptors on the brain epithelium (e.g., transferrin receptor) (see Wu et al., Drug. Metabol. Dispos. 26: 937-9 (1998)).

VI. Combined Use of Anti-CD20 Antibodies with Other Agents or Treatment Modalities

A. Anti-B Cell Antibodies in Combination with Radiation

Radiation alone has not proven to be as effective in treating PCNSL as when it is used in combination with other modalities, such as chemotherapy. One aspect of this invention contemplates treating a subject with a brain lymphoma with an anti-CD20 antibody alone or in combination with another agent or agents (e.g., CHOP) in combination with brain irradiation. The antibodies can be administered before, after or both before and after brain irradiation. For example, whole brain radiotherapy (WBRT) can be administered to the subject, followed by high dose treatment with cytarabine and anti-CD20 antibodies alone or in combination with other anti-B cell antibodies. Preferably 4,000 to 5,000 cGy is administered to a subject. Alternatively, a subject can be treated with 4,000 cGy radiotherapy to the brain and a 2,000 cGy boost to the involved area as discussed in DeAngelis et al., 1997. If ocular involvement exists in the subject, then 3,600 cGy to the eyes may be administered.

Radiation can be administered first, followed by therapy with anti-CD20 alone or in combination with other anti-B cell antibodies. Post radiation administration of anti-CD20 antibodies can be combined with procarbazine, lomustine and vincristine (PCV). Administration of PCV can be performed as described in Chamberlain et al., J. Neuro. Oncol. 14: 271-275 (1992). Alternatively, the antibodies can be combined with cyclophosphamide, doxorubicin, vincristine and prednisone (CHOP) or cyclophosphamide, doxorubicin, vincristine and dexamethasone (CHOD). These antibody and chemotherapy combinations can be administered prior to whole brain radiotherapy. The anti-CD20 antibodies of the invention also can be combined with methotrexate (400 mg/M²), doxorubicin, cyclophosphamide, vincristine, prednisone and bleomycin (MACOP-B) preceding cranial irradiation. The administration of MACOP-B, CHOP and CHOD can be preformed as described in DeAngelis et al., 1997 and the references cited therein.

Alternatively, the anti-CD20 antibodies may themselves be linked to a medically useful isotope. Such radionuclides are discussed in further detail below.

B. Anti-CD20 Antibodies in Combination with Chemotherapy

Another embodiment of the invention is the treatment of brain lymphomas using an anti-B cell antibody, e.g., anti-CD20 antibodies or therapeutically effective fragments thereof in combination with chemotherapeutic agents without radiotherapy.

One example is the administration of an anti-CD20 antibody with high dosage methotrexate. Additional agents can also be administered with this combination. For example, the anti-CD20 antibodies of this invention can be administered with high dosage methotrexate (2.5 g/M²), procarbazine and vincristine with the methotrexate, procarbazine and vincristine administered as described in Freilich et al, Neurology 46: 435-439 (1996). High dosage methotrexate can also be administered as described in Perez-Jaffe et al., Diagn. Cvtopathol. 20: 219-223 (1999)). Alternatively, anti-CD20 antibodies can also be administered with high dosage cytarabine (3 g/M²). The administration of high dosage cytarabine can be performed as described in Strauchen et al., Cancer 63: 1918-21 (1989). Another embodiment of the invention contemplates the combined administration of anti-CD20 antibodies and chemotherapeutics, and/or with anti-CD40 or anti-CD40L antibodies and/or with other anti-B cell antibodies.

C. Anti-B Cell Antibodies Such as Anti-CD20 Antibody in Combination with Agents which Increase Blood Brain Barrier Permeability

As the blood brain barrier can pose a problem for administration of drugs to a patient, the use of agents or methods which increase blood brain barrier (BBB) permeability may be utilized in instances where intrathecal administration is not desired, or if alternative forms of administration of anti-CD20 antibodies are preferred. One example of an agent which increases BBB permeability is an antibody which is reactive with a transferrn receptor present on brain capillary endothelial cells. Monoclonal antibodies which are reactive with at least a portion of the transferrin receptor include: OX-26, B3/25, Tf6/14, OKT-9, L5.1, 5E-9, RI7 217 and T58/30. These anti-transferrin receptor antibodies can be utilized as described in U.S. Pat. No. 5,182,107, which is herein incorporated by reference in its entirety.

The compositions contemplated by the invention may also comprise lipophilic vectors (e.g., procarbazine) for delivery of the antibodies to the target site in the brain. Immunoliposomes are also contemplated (Huwyler et al., 1996). Lipophilic molecules are preferably fatty acids of the omega-3 series or lipid derivatives thereof. Other lipophilic molecules are fatty acids, diacyl glycerols, diacyl phospholipids, lyso-phospholipids, cholesterol, and other steroids, bearing poly-unsaturated hydrocarbon groups of 18 to 46 carbon atoms.

Preferred biopolymer carriers are poly(alpha)-amino acids (e.g., PLL, poly L-5 arginine:PLA, poly L-omithine:PLO), human serum albumin, aminodextran, casein, etc. These carriers preferably are biodegradable, biocompatible and potentially excellent candidates for drug delivery systems. For further description of such carriers and their administration, see U.S. Pat. No. 5,716,614, which is herein incorporated by reference in its entirety.

VII. Administration of Anti-B Cell Antibodies Such as Anti-CD20 Antibody in Combination with Agents Which Interfere with CD40/CD40L Interaction

Another method contemplated by this invention is the treatment of brain lymphomas using a combination of a B cell antibody, preferably a B cell depleting antibody, and most preferably depleting anti-CD20 antibodies with agents which interfere with the CD40/CD40L interaction, preferably anti-CD40 or anti-CD40L antibodies.

According to this aspect of the invention, a “CD40L antagonist” is administered to a subject to interfere with the interaction of CD40L and its binding partner, CD40 in combination with an anti-B cell antibody, e.g. RITUXAN®. A “CD40L antagonist” is defined as a molecule which interferes with this interaction. The CD40L antagonist can be an antibody directed against CD40L (e.g., a monoclonal antibody against CD40L), a fragment or derivative of an antibody against CD40L (e.g., Fab or F(ab)′₂ fragments, chimeric antibodies or humanized antibodies), soluble forms of CD40, soluble forms of a fusion protein comprising CD40, or pharmaceutical agents which disrupt or interfere with the CD40L-CD40 interaction.

To prepare anti-CD40L antibodies, a mammal (e.g., a mouse, hamster, rabbit or ungulate) can be immunized with an immunogenic form of CD40L protein or protein fragments thereof (e.g., peptide fragments), which elicits an antibody response in the mammal. A cell expressing CD40L on its surface can also be utilized as an immunogen. Alternative immunogens include purified CD40L protein or protein fragments. CD40L can be purified from a CD40L-expressing cell by standard purification techniques (Armitage et aL, Nature 357:80-82 (1992); Lederman et al, J. Ex. Med. 175: 1091-1101 (1992); and Hollenbaugh et al., EMBO J. 11:4313-4321 (1992)). Alternatively, CD40L peptides can be prepared based upon the amino acid sequence of CD40L, as disclosed in Armitage et al., (1992). Techniques for conferring immunogenicity on a protein include conjugation to carriers or other techniques well known in the art. For example, the protein can be administered in the presence of an adjuvant. The process of immunization can be monitored by detection of antibody titers in plasma or serum. Standard ELISA or other immunoassays can be used with the immunogen as antigen to assess the levels of antibodies. Following immunization, antisera can be obtained and polyclonal antibodies isolated. To produce monoclonal antibodies, antibody producing cells can be harvested and fused with myeloma cells using standard somatic cell fusion procedures, as described in U.S. Pat. Nos. 5,833,987 (1998) and 5,747,037 (1997). Anti-CD20 and anti-CD40 antibodies can be prepared by similar methods. Several anti-CD40L antibodies anti-CD40 antibodies and anti-CD20 antibodies have been reported in the literature, which are publicly available.

Antibodies can be fragments, and the fragments screened for utility in the same manner as described above for whole antibodies. For example, F(ab′)₂ fragments can be generated by treating antibody with pepsin. The resulting F(ab′)₂ fragments can be treated to reduce disulfide bridges to produce Fab′ fragments. Other antibody fragments contemplated include Fab and scFv.

One method of minimizing recognition of non-human antibodies when used therapeutically in humans, other than general immunosuppression, is to produce chimeric antibody derivatives, i.e., antibody molecules that combine a non-human animal variable region and a human constant region. Chimeric antibody molecules can include, for example, the antigen binding domain from an antibody of a mouse, rat or other species, with human constant regions. Methods for making chimeric antibodies include those references cited in U.S. Pat. No. 5,833,987 (1998).

For human therapeutic purposes, the antibodies specifically reactive with a CD40L protein or peptide can be further humanized by producing human variable region chimeras, in which parts of the variable regions, especially the conserved framework regions of the antigen-binding domain, are of human origin and only the hypervariable regions are of non-human origin. Such altered immunoglobulin molecules may be made by any of several techniques known in the art, (e.g., Teng et al., Proc. Natl. Acad. Sci. U.S.A. 80: 7308-7312 (1983); Kozbor et al., Immunology Today 4: 7279 (1983); Olsson et al., Meth. Enzymol. 92: 3-16 (1982)), and are preferably made according to the teachings of PCT Publication W092/06193 or EP 0239400. Humanized antibodies can be commercially produced by, for example, Scotgen Limited, 2 Holly Road, Twickenham, Middlesex, Great Britain.

Another method of generating specific antibodies, or antibody fragments, reactive against a CD40L protein or peptide is to screen expression libraries encoding inimunoglobulin genes, or portions thereof, expressed in bacteria with a CD40L protein or peptide. For example, complete Fab fragments, V_{H} regions and Fv regions can be expressed in bacteria using phage expression libraries. See for example, Ward et al., Nature 341: 544-546 (1989); Huse et al., Science 246: 1275-1281 (1989); and McCafferty et al., Nature 348: 552-554 (1990). Screening such libraries with, for example, a CD40L peptide, can identify immunoglobulin fragments reactive with CD40L. Alternatively, the SCID-hu mouse (available from Genpharm) can be used to produce antibodies, or fragments thereof.

Methodologies for producing monoclonal antibodies (mAb) directed against CD40L, including human CD40L and mouse CD40L, and suitable monoclonal antibodies for use in the methods of the invention, are described in PCT Pat. Application No. WO 95/06666 entitled “Anti-gp39 Antibodies and Uses Therefor,” the teachings of which are incorporated herein by reference in their entirety. Particularly preferred anti-human CD40L antibodies of the invention are MAbs 24-31 and 89-76, produced respectively by hybridomas 24-31 and 89-76. (These antibodies are cloned as described in U.S. Pat. No. 5,747,037). The 89-76 and 24-31 hybridomas, producing the 89-76 and 24-31 antibodies, respectively, were deposited under the provisions of the Budapest Treaty with the American Type Culture Collection, 10801 University Blvd., Manassas, Va. 20110-2209, on Sep. 2, 1994. The 89-76 hybridoma was assigned ATCC Accession Number HB1713 and the 24-31 hybridoma was assigned ATCC Accession Number HB11712.

Recombinant anti-CD40L antibodies, such as chimeric and humanized antibodies, can be produced by manipulating a nucleic acid (e.g., DNA or cDNA) encoding an anti-CD40L antibody according to standard recombinant DNA techniques. Accordingly, another aspect of this invention pertains to isolated nucleic acid molecules encoding immunoglobulin heavy or light chains, or portions thereof, reactive with CD40L, particularly human CD40L. The immunoglobulin-encoding nucleic acid can encode an immunoglobulin light (V_{L}) or heavy (V_{H}) chain variable region, with or without a linked heavy or light chain constant region (or portion thereof). Such nucleic acids can be isolated from a cell (e.g., hybridoma) producing an anti-human CD40L mAb by standard techniques. For example, nucleic acids encoding the 24-31 or 89-76 mAb can be isolated from the 24-31 or 89-76 hybridomas, respectively, by cDNA library screening, PCR amplification or other standard techniques. Moreover, nucleic acids encoding an anti-human CD40L mAb can be incorporated into an expression vector and introduced into a suitable host cell to facilitate expression and production of recombinant forms of anti-human CD40L antibodies.

The methods described above can be utilized with respect to the preparation of either anti-CD20, anti-CD40L or anti-CD40 antibodies.

In addition to antibodies which recognize and bind to CD40L and inhibit CD40 interaction with CD40, other CD40L antagonists are contemplated for use in treating B-cell lymphomas and leukemias, either alone or in combination with other therapies (e.g., radiation or chemotherapeutics). CD40L antagonists can be soluble forms of a CD40L ligand. A monovalent soluble ligand of CD40L, such as soluble CD40, can bind CD40L, thereby inhibiting the interaction of CD40L with the CD40 on expressed B-cells. The term “soluble” indicates that the ligand is not permanently associated with a cell membrane. A soluble CD40L ligand can be prepared by chemical synthesis, or, preferably by recombinant DNA techniques, for example by expressing only the extracellular domain (absent the transmembrane and cytoplasmic domains) of the ligand. A preferred soluble CD40L ligand is soluble CD40. Alternatively, a soluble CD40L ligand can be in the form of a fusion protein. Such a fusion protein comprises at least a portion of the CD40L ligand attached to a second molecule. For example, CD40 can be expressed as a fusion protein with an immunoglobulin (i.e., a CD40Ig fusion protein). In one embodiment, a fusion protein is produced comprising amino acid residues of an extracellular domain portion of the CD40 molecule joined to amino acid residues of a sequence corresponding to the hinge, C_{H}2 and C_{H}3 regions, of an immunoglobulin heavy chain, e.g., Cα1, to form a CD40Ig fusion protein (see e.g., Linsley et at., J. Exp. Med. 1783: 721-730 (1991); Capon etal., Nature 337: 525-531 (1989); and U.S. Pat. No. 5,116,964(1992)). Such fusion proteins can be produced by chemical synthesis, or, preferably by recombinant DNA techniques based on the cDNA of CD40 (Stamenkovic et al., EMBO J. 8: 1403-1410 (1989)).

A CD40L or a CD40 antagonist is administered to subjects in a biologically compatible form suitable for pharmaceutical administration in vivo. By “biologically compatible form suitable for administration in vivo” is meant a form of the antagonist to be administered in which any toxic effects are outweighed by the therapeutic effects of the protein. The term “subject” is intended to include living organisms in which an immune response can be elicited, e.g., mammals. Examples of preferred subjects include humans, dogs, cats, horses, cows, pigs, goats, sheep, mice, rats, and transgenic species thereof. A CD40L or a CD40 antagonist can be administered in any pharmacological form, optionally in a pharmaceutically acceptable carrier. Administration of a therapeutically effective amount of the CD40L or CD40 antagonist is defined as an amount effective, at dosages and for periods of time necessary to achieve the desired result (e.g., inhibition of the progression or proliferation of the brain lymphoma being treated). For example, a therapeutically active amount of a CD40L antagonist may vary according to factors such as the disease stage (e.g., stage I versus stage IV), age, sex, medical complications (e.g., AIDS) and weight of the subject, and the ability of the antagonist to elicit a desired response in the subject. The dosage regimen may be adjusted to provide the optimum therapeutic response. For example, several divided doses may be administered daily, or the dose may be proportionally reduced as indicated by the exigencies of the therapeutic situation. The active compound, such as an anti-CD40 antibody, by itself or in combination with other active agents, may be administered in a convenient manner such as by injection (subcutaneous, intramuscularly, intrathecal, intraventricular, intravenous, etc.), oral administration, inhalation, transdermal application or rectal administration. Depending on the route of administration, the active compound may be coated in a material to protect the compound from the action of enzymes, acids and other natural conditions that may inactivate the compound. A preferred route of administration is intravenous (i.v.) injection.

To administer a CD40L antagonist or CD40 antagonist by other than parenteral administration, it may be necessary to coat the antagonist with, or co-administer the antagonist with, a material to prevent its inactivation. For example, an antagonist can be administered to an individual in an appropriate carrier or diluent, co-administered with enzyme inhibitors or in an appropriate carrier or vector, such as a liposome. Pharmaceutically acceptable diluents include saline and aqueous buffer solutions. Enzyme inhibitors include pancreatic trypsin inhibitor, diisopropylfluorophosphate (DEP) and trasylol. Liposomes include water-in-oil-in-water emulsions, as well as conventional liposomes (Strejan et al., J Neuroimmunol. 7: 27 (1984)). Additional pharmaceutically acceptable carriers and excipients are known in the art.

The active compound may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injection include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms, such as bacteria and fumgi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating an active compound (e.g., an antagonist of CD40L or CD40 by itself or in combination with other active agents or an anti-CD20 antibody and an anti-B cell antibody) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof.

When the active compound is suitably protected, as described above, the protein may be orally administered, for example, with an inert diluent or an assimilable, edible carrier. As used herein, “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. All compositions discussed above for use with CD40L or CD40 antagonists may also comprise supplementary active compounds (e.g., chemotherapeutic agents) in the composition. Moreover, the pharmaceutical compositions described above may also be utilized in preparing compounds comprising anti-CD20 antibodies.

VIII. Treatment of CNS Using Radioimmunotherapy

For radiolabeling, an the active antibody (e.g., anti-B cell antibodies, etc.) for use as a therapeutic or diagnostic, there are several considerations. First, the radioisotope must be chosen, and then the means of attaching the radioisotope to the antibody must be selected. With respect to the choice of a radioisotope, a general review of considerations is provided by Magerstadt, ANTIBODY CONJUGATES AND MALIGNANT DISEASE, 93-109 (1991). Principally, one must consider the desired range of emission (affected by parameters including tissue type of the tumor, whether it is a solid or disseminated tumor and whether or not all tumor cells are expected to be antigen positive), the rate of energy release, the half-life of the isotope as compared to the infusion time and clearance rate, whether imaging or therapy is the aim of the labeled antibody administration, and the like. For diagnostic imaging purposes according to the present invention, it is considered that labeling with ⁹⁹Tc, ¹¹¹In, ¹²³I or ¹³¹I is preferable, with ¹¹¹In or ¹³¹I labeling being most preferred. For therapeutic purposes according to the present invention, it is considered that labeling with a β-emitter, such as ⁹⁰Y or ¹³¹I,is preferable. Other medically suitable isotopes that merit consideration for therapeutic or diagnostic uses are: ¹⁸⁶Re, ¹⁸⁸Re, ¹⁵³Sm, ²¹²Bi, ³²P, ²¹¹At, ⁶⁷Cu, ²¹²Pb and radioactive isotopes of Lu.

In considering the means for attaching the radioisotope to the antibody, one must consider first the nature of the isotope. Iodine isotopes can be attached to the antibody by a number of methods which covalently attach the isotope directly to the protein. Chloramine T labeling (Greenwood et al., Biochem. J. 89: 114 (1963)) and iodogen labeling (Fraker et al, Biochem. Biophys. Res. Comm. 80: 849-857 (1978))) are two commonly used methods of radioiodine labeling. For isotopes of metals, e.g., ⁹⁰Y or ¹⁸⁶Re, the isotope is typically attached by covalently attaching a chelating moiety to the antibody and then allowing the chelator to coordinate the metal. Such methods are described, for example, by Gansow et al., U.S. Pat. Nos. 4,831,175; 4,454,106 and 4,472,509, each of which are hereby incorporated in its entirety by reference. It should be noted that antibodies labeled with iodine isotopes (e.g., ¹³¹I) are subject to dehalogenation upon internalization into the target cell, while antibodies labeled by chelation are subject to radiation-induced scission of the chelator and thereby loss of radioisotope by dissociation of the coordination complex. In some instances, metal dissociated from the complex can be re-complexed, providing more rapid clearance of non-specifically localized isotope and therefore less toxicity to non-target tissues. For example, chelator compounds such as EDTA or DTPA can be infused into patients to provide a pool of chelator to bind released radiometal and facilitate excretion of free radioisotopes in the urine. Also, it merits noting that free iodine, resulting from dehalogenation, and small, iodinated proteins are rapidly cleared from the body. This is advantageous in sparing normal tissue, including bone marrow, from radiotoxic effects.

Methods of administration are also reviewed by Magerstadt (1991). For treatment of lymphoma, it is considered on the one hand that intravenous injection is a good method, as the thoroughness of the circulation in rapidly distributing the labeled antibody is advantageous, especially with respect to avoiding a high local concentration of the radiolabel at the injection site. Intravenous (iv) administration is subject to limitation by a “vascular barrier,”0 comprising endothelial cells of the vasculature and the subendothelial matrix which also is responsible for the BBB. It is considered well-known to those of skill in the art how to formulate a proper composition of a labeled antibody for any of the aforementioned injection routes.

The timing of the administration can vary substantially. The entire dose can be provided in a single bolus. Alternatively, the dose can be provided by an extended infusion method or by repeated injections administered over a span of weeks. A preferable interval of time is six to twelve weeks between radioimmunotherapeutic doses. If low doses are used for radioimmunotherapy, the agent could be administered at two week intervals. If the total therapeutic dose is fractionally delivered, it could be administered over a span of 2 to 4 days. Due to the lower dose infused, trace-labeled doses can be administered at short intervals; for clinical purposes, one to two week intervals are preferred.

The radiometric dosage to be applied can vary substantially. For immunodiagnostic imaging, trace-labeling of the antibody is used, typically about 1-20 mg of antibody is labeled with about 1 to about 35 mCi of radioisotope. The dose is somewhat dependent upon the isotope used for imaging; amounts in the higher end of the range, preferably about 20 to about 30 mCi, should be used with ^{99m}Tc and ¹²³I; amounts in the lower end of the range, preferably about 1-10 mCi, should be used with ¹³¹I and ¹¹¹In. For imaging purposes, about 1 to about 30 mg of such trace-labeled antibody is given to the subject. For radioimmunotherapeutic purposes, the antibody is labeled to high specific activity. The specific activity obtained depends upon the radioisotope used; for ¹³¹I, activity is typically 1 to 10 mCi/mg. The antibody is administered to the patient in sufficient amounts that the whole body dose received is up to 1,100 cGy, but preferably less than or equal to 500 cGy. The amount of antibody, including both labeled and unlabeled antibody, can range from about 0.2 to about 40 mg/kg of patient body weight. Either labeled anti-CD20 or anti-CD40 can be used to diagnose or determine localization of PCNSL or other brain lymphoma.

An amount of radioactivity which would provide approximately 500 cGy to the whole body is estimated to be about 825 mCi of ¹³¹I. The amounts of radioactivity to be administered again depend, in part, upon the isotope chosen. For therapeutic regimens using ¹³¹I, about 5 to about 1,500 mCi might be employed, with preferable amounts being about 5 to about 800 mCi, and about 5 to about 250 mCi being most preferable. For ⁹⁰Y therapy, about 1 to about 200 mCi amounts of radioactivity are considered appropriate, with more preferable amounts being about 1 to about 150 mCi, and about 1 to about 100 mCi being most preferred. The preferred means of estimating tissue doses from the amount of administered radioactivity is to perform an imaging or other pharmacokinetic regimen with a tracer dose, so as to obtain estimates of predicted dosimetry.

Either or both the diagnostic and therapeutic administrations can be preceded by “pre-doses” of unlabeled antibody. The effects of pre-dosing upon both imaging and therapy have been found to vary from patient to patient. Generally, it is preferable to perform a series of diagnostic imaging administrations, using increasing pre-doses of unlabeled antibody. Then the pre-dose providing the best ratio of tumor dose to whole body dose is used prior to the administration of the radioimmunotherapeutic dose.

Goldberg et al describe radioimmunodiagnostic imaging and radioimmunotherapy of solid tumors (carcinomas) using an anti-carcinoembryonic (CEA) antigen antibody (J. Clin. Oncol. 9: 548 (1991)). Many aspects of the materials and methods described in U.S. Pat. Nos. 4,348,376 and 4,460,559, hereby incorporated in their entirety by reference, also can be applied to the present invention, which is directed to the diagnosis and therapy of cerebral lymphomas. Additional description of methods for estimating the radiometric dose received by a patient are provided in reference (Siegel et al., Med. Phys. 20: 579-582 (1993)).

IX. Pharmaceutical Compositions

Conjugation or linkage of the anti-B cell antibody (e.g., anti-CD20, anti-CD22, anti-CD21, anti-CD40 or anti-CD40L antibodies or fragments thereof) of the present invention to the detectable marker or therapeutic agent can be by covalent or other chemical binding means. The chemical binding means can include, for example, glutaraldehyde, heterobifinctional, and homobifuictional linking agents. Heterobifunctional linking agents can include, for example, SMPT (succinimidyl oxycarbonyl-a-methyl-a-(2-pyridyldition)-tolume), SPDP (N-succinimidyl-3-(2-pyridylilithio) propionate) and SMCC (succinimidyl-4-(N-male-imidomethyl) cyclohexane-1-carboxylate). Homobifunctional linking agents can include, for example, DMP (dimethyl pimelimidate), DMA (dimethyl suberinidate) and DTBP (dimethyl 3,3′-dithio-bispropionimidate).

Certain protein detectable markers and therapeutic agents can be recombinantly combined with the variable regions of the monoclonal antibodies of the present invention to construct compositions which are fusion proteins, wherein the monoclonal antibody variable regions maintain their binding specificity and the detectable marker or therapeutic agent retains their activity. Recombinant methods to construct these fusion proteins are well known in the art.

Pharmaceutical compositions comprising monoclonal antibody or recombinant binding proteins, either conjugated or unconjugated, are encompassed by the present invention. A pharmaceutical composition can comprise the monoclonal antibody and a pharmaceutically acceptable carrier. For the purposes of the present invention, a “pharmaceutically acceptable carrier” can be any of the standard carriers well known in the art. For example, suitable carriers can include phosphate buffered saline solutions, emulsions such as oil/water emulsions, and various types of wetting agents. Other carriers can also include sterile solutions, tablets, coated tablets, and capsules. Typically, such carriers can contain excipients such as starch, milk, sugar, types of clay, gelatin, stearic acid, or salts thereof, magnesium or calcium sterate, talc, vegetable fats or oils, gums, glycerols, or other known excipients. Such carriers can also include flavors and color additives, preservatives, or other ingredients. Compositions comprising such carriers are formulated by well known conventional means. See REMINGTON'S PHARMACEUTICAL SCIENCE (15th ed. 1980).

For diagnostic purposes, the antibodies and recombinant binding proteins can be either labeled or unlabeled. Typically, diagnostic assays entail detecting the formation of a complex through the binding of the monoclonal antibody or recombinant binding protein to the human CD20 either at the cell surface. When unlabeled, the antibodies and recombinant binding proteins find use in agglutination assays. In addition, unlabeled antibodies can be used in combination with other labeled antibodies (second antibodies) that are specifically reactive with the monoclonal antibody or recombinant binding protein, such as antibodies specific for immunoglobulin. Alternatively, the monoclonal antibodies and recombinant binding proteins can be directly labeled. A wide variety of labels can be employed, such as radionuclides, (discussed above) fluorescers, enzymes, enzyme substrates, enzyme cofactors, enzyme inhibitors, ligands (particularly haptens), etc. Numerous types of immunoassays are well known in the art.

Commonly, the monoclonal antibodies and recombinant binding proteins of the present invention are used in fluorescent assays, where the subject antibodies or recombinant binding proteins are conjugated to a fluorescent molecule, such as fluorescein isothiocyanate (FITC).

The examples provided below are not meant to limit the invention in any way, but serve to provide preferred embodiments for the invention.

### EXAMPLES

### Example 1

### Intrathecal Rituximab in Non-Human Primates

As meningeal relapse is a common site of recurrence in patients with lymphoma, the use of Rituximab may be beneficial in preventing or inhibiting onset of meningeal relapse.

Materials and Methods.

A continuously maintained non-human primate model has been approved by the NCI, which has a chronically indwelling Pudenz 4^{th} ventricular catheter attached to a subcutaneous Ommaya reservoir. The catheter allows for sampling of the cerebrospinal fluid (CSF) at multiple time points in unanesthetized animals (see McCully et aL, Lab. Animal Sci. 40: 520-525 (1990)).

Doses of Rituximab up to 10 mg are administered at full strength (10 mg/ml) or diluted up to 1 ml in sterile saline without preservative. A sample of the dilute drug solution is saved for later analysis of Rituximab concentration.

The animals used are four adult male rhesus monkeys (Macaca mulatta) weighing approximately 10 kg. The animals are maintained on NIH Open Formula Extruded Non-Human Primate Diet, which is fed to the animals twice daily. Animal #1 (lacking CSF access devices) is injected with an intralumbar injection of Rituximab through a temporary lumbar catheter. Three additional animals shall receive doses of Rituximab in the lateral ventricle via a subcutaneous access device if Animal #1 tolerates the administration of Rituximab. Samples from these animals are obtained from the 4^{th} ventricular Ommaya reservoir, and, in at least one animal, also from the lumbar space. The Ommaya reservoir is pumped four times before and after each CSF sample collection to ensure adequate mixing with ventricular CSF. Two animals with Ommaya reservoirs are also to have 4^{th} ventricular CSF sampling after an intralumbar dose of Rituximab to assess the distribution of the drug from the lumbar space to the ventricle. Once the pharmacokinetic studies have been completed, the tolerance of intrathecal Rituximab is assessed by injecting weekly intralumbar doses more than 6 weeks, in three animals.

CSF pharmacokinetics of Rituximab is studied in four animals following an intrathecal or intraventricular dose of up to 10 mg. CSF samples (0.3 ml) are collected prior to the dose, and again at 0.5, 1, 2, 3, 4, 6, 8, 10 and 24 hours after administration of Rituximab. These samples are frozen immediately at −70□ C and are stored frozen in polypropylene tubes.

### Example 2

### Rituximab Administration into the Cerebrospinal Fluid in the Treatment of Primary CNS Lymphoma in a Rat Model

Materials and Methods.

Toxicity is evaluated in nude rats without tumors, which receive escalating doses of antibody delivered by cisternal puncture. Rituximab (10 mg/ml) is administered to a rat in a volume of 5-100 μl (the CSF volume of the rat is approximately 1 ml). Assuming no toxicity, efficacy studies will then be conducted. B-lymphoid tumor cells with documented anti-CD20 sensitivity are implanted into the cisterna magna of a rat. Animals are then divided into two groups of ten: control and Rituximab treatment at one week post tumor implantation. The end points are the measurement of neurologic performance, weight loss, survival and morphometric and histologic correlates of anti-lymphoma activity.

### Example 3

### Testing of Rituximab in Human Patients with PCNSL

Materials and Methods.

Rituximab is administered as an injection of 5-10 ml into an Omraya reservoir. Before injection, an equivalent volume of CSF is removed to minimize significant flux in CSF volume (the mean volume of CSF in adults is 104 ml). No other chemotherapy or radiotherapy is administered. Treatments consist of injecting Rituximab in a volume of 5-10 ml into an Ommaya reservoir. CSF and serum levels of Rituximab are measured at 1, 2, 4, 24, 48, 72 hours and 7 days and at regular intervals thereafter.

Patients with relapsed PCNSL must be CD20⁺on pathologic analysis. The patient must be older than 17 years, have a KPS less than 50, have a life expectancy of less than 2 months, have systemic involvement of PCNSL, and cannot have received radiation or chemotherapy less than 5 weeks before initiation of intra-CSF administration of Rituximab.

Study patients are divided into groups of three, each group receiving a given dose level of Rituximab through an Ommaya reservoir. One week later, Rituximab administration is repeated into the CSF begins at an interval determined by the calculated clearance in primates. Rituximab administration proceeds for 90 days, during which there is an on-going evaluation of toxicity and response. Early termination will be mandatory for any grade four neurotoxicity attributed to intra-CSF administration of Rituximab. Neurotoxicity is the basis for evaluating safety and determining if the study should be stopped or a lower dosage utilized. Assuming no toxicity is evident at the given dose level, the dose is then to be escalated to the next level. The goal is to determine a safe dose which achieves trough levels of Rituximab in CSF at least ten times greater than the serum trough levels associated with activity in humans (McLaughlin et al., J. Clin. Oncol. 16: 2825-2833 (1998)).

### Example 4

### Method of Administering Rituximab with Methotrexate in a Human Subject to Treat PCNSL

A patient with CNS involvement with lymphoma can be treated with intrathecal methotrexate (15 mg) in combination with Rituximab at dosages ranging from 250 mg/M² weekly times four to 350 mg/M² weekly times four.

### Example 5

### Method of Treating PCNSL with Radioactively Labeled Rituximab and CHOP

A patient with PCNSL can be treated with radioactively labeled Rituximab and the chemotherapy combination CHOP (e.g., cyclophosphamide, doxorubicin vincristine and prednisone) as follows. The CHOP therapy would be administered intravenously according to standard procedures. Rituximab labeled with 131 -Iodine is administered to the subject intrathecally at a dosage of about 1 to about 10 mCi., with the amount of Rituximab (both labeled and unlabeled) ranging from about 0.2 to about 40 mg/kg of patient body weight. The radioactive Rituximab can be administered either in a single bolus or over a period of about 2 to about 4 days.

All references described above are herein incorporated by reference in their entirety.

Claims:

1. A method of treating a central nervous system (CNS) lymphoma comprising the step of administering a therapeutically effective amount of an anti-CD20 antibody or fragment thereof.

2. A method to treat or prevent meningeal relapse in a subject with lymphoma comprising the step of administering a therapeutically effective amount of an anti-CD20 antibody or fragment thereof.

3. The method of claim 1, wherein the CNS lymphoma is selected from the group consisting of: primary CNS lymphoma, (PCNSL) leptomeningeal metastasises (LM), or Hodgkin's Disease with CNS involvement.

4. The method of claim 3, wherein the CNS lymphoma is LM and wherein the anti-CD20 antibody or fragment thereof is administered in combination with cytarabine and thiotepa or methotrexate and ¹¹¹In-diethylenetriamine pentaacetic acid.

5. The method of claim 1, wherein the anti-CD20 antibody fragment is selected from the group consisting of Fab, Fab′ and F(ab′)₂.

6. The method of claim 2, wherein the anti-CD20 antibody fragment is selected from the group consisting of Fab, Fab′ and F(ab′)₂.

7. The method of claim 1, wherein the anti-CD20 antibody is a human antibody, humanized, bispecific or chimeric.

8. The method of claim 2, wherein the anti-CD20 antibody is a human antibody, humanized, bispecific or chimeric.

9. The method of claim 1, wherein the anti-CD20 is Rituximab or IF5.

10. The method of claim 2, wherein the anti-CD20 is Rituximab or IF5.

11. The method of claim 9, wherein the anti-CD20 antibody is Rituximab and is administered to the subject in a dosage of about 10 mg to about 375 mg/M² per week for four weeks.

12. The method of claim 11, wherein the anti-CD20 antibody is Rituximab and is administered to the subject in a dosage of about 10 mg to about 375 mg/M² per week for four weeks.

13. The method of claim 1, wherein the anti-CD20 antibody is administered intrathecally or intraventrically.

14. The method of claim 2, wherein the anti-CD20 antibody is administered intrathecally or intraventrically.

15. The method of claim 1, wherein the anti-CD20 antibody is administered in combination with methotrexate, CHOP, CHOD cytarabine, leucovorin, thiotepa and vincristine or combinations thereof.

16. The method of claim 2, wherein the anti-CD20 antibody is administered in combination with methotrexate, CHOP, CHOD cytarabine, leucovorin, thiotepa and vincristine or combinations thereof.

17. The method of claim 1, wherein the anti-CD20 antibody is administered prior to whole brain irradiation.

18. The method of claim 1, wherein the anti-CD20 antibody is Rituximab and is administered intrathecally with methotrexate.

19. The method of claim 1, wherein the anti-CD20 antibody is Rituximab and the antibody is labeled.

20. The method of claim 19, wherein Rituximab is labeled with an isotope selected from the group consisting of: ²¹¹At, ²¹²Bi, ⁶⁷Cu, ¹²³I, ¹³¹I, ¹¹¹In, ³²p, ²¹²Pb, ¹⁸⁶Re, ¹⁸⁸Re, ¹⁵³Sm, ^{99m}Tc, and ⁹⁰Y and is administered in a radioimmunotherapeutically effective amount.

21. The method of claim 20, wherein the radioimmunotherapeutically effective amount provides irradiation at a dose in the range of about 10 to about 200 cGy to the whole body of the patient.

22. The method of claim 22, wherein the anti-CD20 antibody is administered in combination with an anti-CD40 antibody or an agent which inhibits interaction of CD40 with CD40L.

23. The method of claim 22, wherein the anti-CD20 antibody is administered in a pharmaceutically acceptable dosage of the antibody ranging from about 0.001 to about 30 mg/kg of human body weight.

24. The method of claim 23, wherein the anti-CD20 antibody is administered in a pharmaceutically acceptable dosage of the antibody ranging from about 0.01 to about 25 mg/kg human body weight.

25. The composition of claim 24, wherein the anti-CD20 antibody is administered in a pharmaceutically acceptable dosage of the antibody ranging from about 0.4 to about 20.0 mg/kg human body weight.

26. A method of diagnosing PCNSL in a subject comprising the steps of:
(A) administering to said subject an anti-CD20 antibody or anti-CD20 antibody fragment bound to a detectable label; and
(B) detecting the localization of said label.

27. The method of claim 26, wherein the detectable label is: ²¹¹At,²¹²Bi, ⁶⁷Cu, ¹²³I, ¹³¹I, ¹¹¹In, ³²p, ²¹²Pb, ¹⁸⁶Re, ¹⁸⁸Re, ¹⁵³Sm, ^{99m}Tc, or ⁹⁰Y.

28. The method of claim 26, wherein the anti-CD20 antibody is Rituximab.

29. The method of claim 1, wherein the anti-CD20 antibody is linked to a brain blood barrier (BBB) permeability enhancing agent.

30. The method of claim 29, wherein the BBB permeability enhancing agent is OX-26, B3/25, Tf6/14, OKT-9, L5.1, 5E-9, RI7 217 or T58/30.

31. The method of claim 1, wherein the anti-CD20 antibody further comprises a lipophilic vector or an immunolipophilic vector.

32. The method of claim 31, wherein the lipophilic vector is procarbazine, an omega-3 fatty acid, a diacyl glycerol, a diacyl phospholipid, a lyso-phospholipid, cholesterol or a steroid.

33. The method of claim 1, further comprising the step of administering an anti-B cell antibody or fragment thereof in combination with the anti-CD20 antibody or fragment thereof.

34. The method of claim 33, wherein the anti-B cell antibody is anti-CD19 antibody or fragments thereof, anti-CD22 antibody or fragments thereof, anti-CD38 antibody or fragments thereof, or anti-major histocompatibility complex (MHC) II antibody or fragments thereof.

35. A composition for the treatment of CNS lymphoma for intrathecal administration comprising an anti-CD20 antibody and an anti-B cell antibody wherein the antibodies are administered at a dosage ranging from about 0.4 to about 20.0 mg/kg human body weight.

36. A method of treating a central nervous system (CNS) lymphoma comprising intrathecally administering a therapeutically effective amount of an antibody or antibody fragment that binds to a B cell antigen.

37. The method of claim 36 wherein said antigen is selected from the group consisting of CD10, CD14, CD20, CD21, CD22, CD23, CD24, CD37, CD53, CD72, CD73, CD74, CD75, CD76, CD77, CD78, CD79a, CD79b, CD80, CD81, CD82, CD83, CDw84, CD85 and CD86.

38. The method of claim 36 wherein said antibody is a B cell depleting antibody.

39. The method of claim 36 wherein said antibody or antibody fragment is conjugated to a toxin.

40. The method of claim 36 wherein said antibody or antibody fragment is conjugated to a drug.

41. The method of claim 36 wherein said antibody or antibody fragment is conjugated to an enzyme.

42. The method of claim 36 wherein said antibody or antibody fragment is conjugated to a radionuclide.

43. The method of claim 36 wherein said antibody or antibody fragment is administered in combination with at least one chemotherapeutic.

44. The method of claim 43 wherein said chemotherapeutic is selected from the group consisting of thiotepa, cyclosphosphamide, busulfan, improsulfan, piposulfan, benzodopa, carboquone, meturedopa, uredopa, altretamine, triethylenemelamine, trietylenephosphoramide, triethylenethiophosphaoramide, trimethylolomelamine, chlorambucil, chlornaphazine, cholophosphamide, estramustine, ifosfamide, mechlorethamine, mechlorethamine oxide hydrochloride, melphalan, novembiehin, phenesterine, prednimustine, trofosfamide, uracil mustard, carmustine, chlorozotocin, fotemustine, lomustine, nimustine, ranimustine, aclacinomysins, actinomycin, authramycin, azaserine, bleomycins, cactinomycin, calicheamicin, carabicin, carminomycin, carzinophilin, chromoinycins, dactinomycin, daunorubicin, detorubicin, 6-diazo-5-oxo-L-norleucine, doxorubicin, epirubicin, esorubicin, idambicin, marcellomycin, mitomycin, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, 5-FU, calusterone, dromostanolone propionate, epitiostanol, mepitiostane, testolactone, aminoglutethimide, mitotane, trilostane, frolinic acid, aceglatone, aldophosphamide glycoside, aminolevulinic acid, amsacrine, bestrabucil, bisantrene, edatraxate, defofamine, demecolcine, diaziquone, elfornithine, elliptinium acetate, etoglucid, gallium nitrate, hydroxyurea, lentinan, lonidamine, mitoguazone, mitoxantrone, mopidamol, nitracrine, pentostatin, phenamet, pirarubicin, podophyllinic acid, 2-ethylhydrazide, procarbazine, razoxane, sizofrran, spirogermanium, tenuazonic acid, triaziquone, 2,2′,2″-trichlorotriethylamine, urethan, vindesine, dacarbazine, mannomustine, mitobronitol, mitolactol, pipobroman, gacytosine, arabinoside, cyclophosphamide, thiotepa, paclitaxel, doxetaxel, chlorambucil, gemcitabine, 6-thioguanine, mercaptopurine, methotrexate, cisplatin, carboplatin, vinblastine, platinum, etoposide (VP-16), ifosfamide, mitomycin C, mitoxantrone, vincristine, vinorelbine, navelbine, novantrone, teniposide, daunomycin, aminopterin, xeloda, ibandronate, topoisomerase inhibitor, difluoromethylornithine (DMFO), retinoic acid, esperamicins, capecitabine, tamoxifen, raloxifene, aromatase inhibiting 4(5)-imidazoles, 4 hydroxytamoxifen, trioxifene, keoxifene, LY117018, onapristone, toremifene, flutamide, nilutamide, bicalutamide, leuprolide, goserelin; and pharmaceutically acceptable salts, acids or derivatives of any of the above.

45. The method of claim 36 wherein said antibody or antibody fragment is specific to a B cell antigen selected from the group consisting of CD19, CD20, CD21, CD22, CD37 and CD40.

46. The method of claim 45 wherein said antibody or antibody fragment is RITUXAN® and said method of treatment further comprises administration of a cytokine.

47. The method of claim 46 wherein said cytokine is IL-10.

48. The method of claim 36 which comprises administration of a depleting anti-CD20 antibody and a CD40L antagonist.

49. The method of claim 48 wherein said CD40L ant agonist is an antibody th at specifically binds CD40L.

50. The method of claim 36 wherein a radiolabeled antibody to CD20 is administered.
